(19) United States
(12) Patent Application Publication
(10) Pub. No.: US 2023/0333120 A1
(43) Pub. Date: Oct. 19, 2023

(54) A Newborn Metabolic Vulnerability Model For Identifying Preterm Infants At Risk Of Adverse Outcomes, And Uses Thereof

(71) Applicants: The Regents of the University of California, Oakland, CA (US); University of Iowa Research Foundation, Iowa City, IA (US)

(72) Inventors: Laura Jelliffe-Pawlowski, San Francisco, CA (US); Scott P. Oltman, San Francisco, CA (US); Kelli K. Ryckman, Iowa City, IA (US)

(21) Appl. No.: 18/025,104

(22) PCT Filed: Sep. 8, 2021
(86) PCT No.: PCT/US2021/049513

§ 371 Date: Mar. 7, 2023

Related U.S. Application Data
(60) Provisional application No. 63/075,807, filed on Sep. 8, 2020.

(51) Int. Cl. G01N 33/68; G01N 33/76; G01N 33/74; G01N 33/92; G01N 33/573; G16H 50/30

(52) U.S. Cl. CPC G01N 33/6893; G01N 33/76; G01N 33/743; G01N 33/92; G01N 33/573; G16H 50/30; G01N 2800/368; G01N 2800/50; G01N 2333/91245; G01N 2333/59; G16H 50/20

(57) ABSTRACT

The disclosure provides for a newborn metabolic vulnerability profile that can be used to evaluate risk for neonatal mortality and major morbidity in preterm infants, methods of using said model for precision clinical monitoring and targeted investigation of etiologic pathways to reduce the incidence and severity of major morbidities associated with preterm birth.

### CROSS REFERENCE TO RELATED APPLICATIONS

This application claims priority under 35 U.S.C. § 119 from Provisional Application Ser. No. 63/075,807, filed Sep. 8, 2020, the disclosures of which are incorporated herein by reference.

### TECHNICAL FIELD

The disclosure provides for a newborn metabolic vulnerability profile that can be used to evaluate risk for neonatal mortality and major morbidity in infants including pre-term infants, methods of using said model for precision clinical monitoring and targeted investigation of etiologic pathways to reduce the incidence and severity of major morbidities associated with infants and preterm birth.

### BACKGROUND

Worldwide, more than 15 million babies are born preterm (before 37 completed weeks of gestation) each year. Preterm birth (PTB) and its related complications are the leading cause of death in children less than five years of age and contribute to more than 1 million deaths per year. Survivors of PTB are more likely to suffer from both short- and long-term morbidities including blindness, deafness, neurodevelopmental delay, psychiatric disturbance, and diabetes and heart disease in later life. Identifying preterm infants at risk for mortality or major morbidity traditionally relies on gestational age, birthweight, and other clinical characteristics that offer underwhelming utility.

Sudden infant death syndrome (SIDS) is the sudden unexplained death of a child of less than one year of age. SIDS usually occurs during sleep. Typically death occurs between the hours of 00:00 and 09:00. There is usually no noise or evidence of struggle. SIDS remains the leading cause of infant mortality in Western countries, contributing to half of all post-neonatal deaths. The exact cause of SIDS is unknown.

### SUMMARY

A newborn metabolic vulnerability profile that could identify preterm infants at risk for major morbidity and mortality was developed herein. The model was established from a population-based retrospective cohort study of preterm infants born between 2005 and 2011 in California. 9,639 (9.2%) preterm infants experienced mortality or at least one complication. Six characteristics and 19 metabolites were included in the final metabolic vulnerability model. The model demonstrated exceptional performance for the composite outcome of mortality or any major morbidity (AUC 0.923 (95% CI: 0.917-0.929). Performance was maintained across mortality and morbidity subgroups (AUCs 0.893-0.979). The newborn metabolic vulnerability profile disclosed herein out performed models currently in use that rely primarily on infant characteristics.

In a particular embodiment, the disclosure provides a newborn metabolic vulnerability profile model that can be used to evaluate risk for neonatal mortality and major morbidity in preterm infants, comprising: measuring the concentration of one or more metabolites from a sample obtained from a preterm infant, wherein the one or more metabolites are selected from thyroid stimulating hormone (TSH), galactose 1-phosphate uridylyltransferase (GALT), 17-hydroxyprogesterone (17-OHP), 5-oxoproline, glycine, leucine/isoleucine, ornithine, phenylalanine, proline, tyrosine, C-2 acylcarnitine, C-3 acylcarnitine, C-4 acylcarnitine, C-5 acylcarnitine, C-10 acylcarnitine, C-12 acylcarnitine, C-12:1 acylcarnitine, C-16:1 acylcarnitine, and C-18:2 acylcarnitine; assigning a risk indicator value or predictor for each of the measured metabolites based from International Classification of Diseases 9th revision (ICD-9) diagnostic codes, linked infant death records, and/or hospital discharge status; inputting the obtained risk indicator values into a computer implemented multivariable metabolic vulnerability regression model that is built using a training set and a testing set from a population of infants with any mortality or major morbidity to healthy infants; and determining whether the preterm infant has an increased risk for morbidity or mortality based upon the morbidity/mortality predictive value generated from the metabolic vulnerability regression model. In a further embodiment, the sample is obtained from a preterm infant that is born at a gestation age of 32-36 weeks. In another embodiment, the sample is obtained from a preterm infant that is born at a gestation age of under 32 weeks. In a certain embodiment, the sample is a serum or blood sample. In another embodiment, the one or more metabolites are measured using tandem mass spectrometry (MS/MS), high-performance liquid chromatography, and/or a fluorometric enzyme assay. In another embodiment, the metabolic vulnerability profile model further includes risk indicator values or predictor values for one or more characteristics selected from sex of preterm infant, cesarean delivery, maternal education, maternal race/ethnicity, gestational age, and birthweight. In yet another embodiment, the preterm infant at higher risk for a morbidity selected from patent ductus arteriosus (PDA), respiratory distress syndrome (RDS), intraventricular hemorrhage (IVH), periventricular leukomalacia (PVL), bronchopulmonary dysplasia (BPD), retinopathy of prematurity (ROP), necrotizing enterocolitis (NEC), jaundice, infections, sepsis, longer term, cerebral palsy, and/or neurodevelopmental disability. In a further embodiment, the preterm infant is at a higher risk for morbidity or mortality if there is an increased measured concentration for phenylalanine, glycine, 17-OHP, proline, C-4 acylcarnitine, and C-5 acylcarnitine, and a decreased measured concentration for TSH, GALT, 5-oxoproline, ornithine, tyrosine, C-2 acylcarnitine, and C-12 acylcarnitine. In yet a further embodiment, the preterm infant is at a higher risk for morbidity or mortality if there is an increased measured concentration for 17-OHP, glycine, proline, and C-4 acylcarnitine and a decreased measured concentration for TSH, GALT, 5-oxoproline, ornithine, and C-2 acylcarnitine. In a certain embodiment, the method further comprises clinical monitoring and investigating etiologic metabolic pathways of the preterm infant that are related or give rise to the morbidity/mortality predictive value generated from the metabolic vulnerability regression model.

The disclosure provides a method of generating a risk assessment score for a biological sample obtained from a newborn infant, comprising measuring the level of a panel of metabolites in the sample, wherein the panel of metabolites comprises two or more the group consisting of thyroid stimulating hormone (TSH), galactose 1-phosphate uridylyltransferase (GALT), 17-hydroxyprogesterone (17-OHP), 5-oxoproline, glycine, leucine/isoleucine, ornithine, phenylalanine, proline, tyrosine, C-2 acylcarnitine, C-3 acylcarnitine, C-4 acylcarnitine, C-5 acylcarnitine, C-10 acylcarnitine, C-12 acylcarnitine, C-12:1 acylcarnitine, C-16:1 acylcarnitine, and C-18:2 acylcarnitine; assigning a risk indicator value or predictor for each of the measured metabolites; inputting the obtained risk indicator value into a computer-implemented predicative multivariate logistic model that is built using a training set and a testing set from a population of infants with any mortality or major morbidity to healthy infants; and calculating a risk assessment score for the biological sample obtained from the newborn infant using the predicative multivariate logistic model. In one embodiment, the newborn infant is a preterm infant. In a further embodiment, the sample is obtained from a preterm infant that is born at a gestation age of 32-36 weeks. In still another embodiment, the sample is obtained from a preterm infant that is born at a gestation age of under 32 weeks. In another embodiment, the newborn infant is a full-term infant. In yet another embodiment, the sample is a serum or a blood sample. In still another embodiment, of any of the foregoing embodiments, the one or more metabolites are measured using tandem mass spectrometry (MS/MS), high-performance liquid chromatography, and/or a fluorometric enzyme assay. In still another embodiment of any of the foregoing, the predicative multivariate logistic model further includes risk indicator values or predictor values for one or more characteristics selected from sex of preterm infant, cesarean delivery, maternal education, maternal race/ethnicity, gestational age, and birthweight. In another embodiment, the preterm infant is at higher risk for a morbidity selected from patent ductus arteriosus (PDA), respiratory distress syndrome (RDS), intraventricular hemorrhage (IVH), periventricular leukomalacia (PVL), bronchopulmonary dysplasia (BPD), retinopathy of prematurity (ROP), necrotizing enterocolitis (NEC), jaundice, infections, sepsis, longer term, cerebral palsy, and/or neurodevelopmental disability. In yet another embodiment, the infant is at higher risk for Sudden Infant Death Syndrome (SIDS). In still another embodiment, the preterm infant is at a higher risk for morbidity or mortality if there is an increased measured concentration for phenylalanine, glycine, 17-OHP, proline, C-4 acylcarnitine, and C-5 acylcarnitine, and a decreased measured concentration for TSH, GALT, 5-oxoproline, ornithine, tyrosine, C-2 acylcarnitine, and C-12 acylcarnitine. In another embodiment, the preterm infant is at a higher risk for morbidity or mortality if there is an increased measured concentration for 17-OHP, glycine, proline, and C-4 acylcarnitine and a decreased measured concentration for TSH, GALT, 5-oxoproline, ornithine, and C-2 acylcarnitine. In yet another embodiment, the panel of metabolites are measured using a quantitative multiplex assay. In a further embodiment, the quantitative multiplex assay is a quantitative bead-based multiplex immunoassay. In yet another or further embodiment of any of the foregoing embodiments, the predicative multivariate logistic model is a linear discriminant analysis model. In a further embodiment, the linear discriminant analysis model uses the coefficients for the biomarkers presented in Table 2 or 9. In another embodiment, the predictive multivariate logistic model uses the coefficients for the biomarkers presented in Table 2 or 9. In a further embodiment the method further comprises clinical monitoring and investigating etiologic metabolic pathways of the preterm infant that are related or give rise to the morbidity/mortality predictive value generated from the metabolic vulnerability regression model.

The disclosure also provides a kit for assessing preterm birth and preeclampsia risk biomarkers in a sample, wherein the kit comprises a detecting agent(s) for each metabolite in a panel of metabolites consisting essentially of thyroid stimulating hormone (TSH), galactose 1-phosphate uridylyltransferase (GALT), 17-hydroxyprogesterone (17-0HP), 5-oxoproline, glycine, leucine/isoleucine, ornithine, phenylalanine, proline, tyrosine, C-2 acylcarnitine, C-3 acylcarnitine, C-4 acylcarnitine, C-5 acylcarnitine, C-10 acylcarnitine, C-12 acylcarnitine, C-12:1 acylcarnitine, C-16:1 acylcarnitine, and C-18:2 acylcarnitine. In another embodiment, the detecting agents are antibodies. In still a further embodiment, the kit is an ELISA or antibody microarray.

### DESCRIPTION OF DRAWINGS

FIG. 1 provides a flow chart of infants in California between 2005 and 2011 eligible for analysis.

FIG. 2 presents ROC curves of the full model, metabolites only, and characteristics only for any mortality or morbidity in infants of all gestational ages (Left); gestational ages 32-36 (Center); gestational ages<32 (Right).

FIG. 3 demonstrates the importance of individual variables fluctuated within gestational age stratifications and by outcome. Reference for race/ethnicity is white and the reference for education is >12 years. For continuous variables, risk/protective effect corresponds to increasing amounts of the variable. MM: any mortality or morbidity; NM: neonatal mortality; 1-yr M: 1-year mortality; RDS: respiratory distress syndrome; PDA: patent ductus arteriosus; ROP: retinopathy of prematurity; IVH: intraventricular hemorrhage; BPD: bronchopulmonary dysplasia; NEC: necrotizing enterocolitis; PVL periventricular leukomalacia; BW: birthweight; GA: gestational age; Edu: education; HS: high school; GALT: galactose-1-phosphate uridyl transferase; TSH: thyroid stimulating hormone; 17-OHP: 17 hydroxyprogesterone; LEU: leucine/isoleucine ratio.

FIG. 4 shows ROC curves for the full model and subset models of SIDS.

### DETAILED DESCRIPTION

As used herein and in the appended claims, the singular forms “a,” “an,” and “the” include plural referents unless the context clearly dictates otherwise. Thus, for example, reference to “a cytokine” includes a plurality of such cytokines and reference to “the biomarker” includes reference to one or more biomarkers and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the disclosure, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

As used herein, the term “amount” or “level” in reference of a metabolite, refers to a quantity of the metabolite that is detectable or measurable in a biological sample and/or control.

As used herein, the term “biological sample” includes any sample that is taken from a preterm subject which contains one or more metabolites described herein. Suitable samples in the context of the present disclosure include, for example, blood, plasma, serum, amniotic fluid, vaginal excretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles.

As used herein, the terms “isolated” and “purified,” generally describes a composition of matter that has been removed from its native environment (e.g., the natural environment if it is naturally occurring), and thus is altered by the hand of man from its natural state. An isolated protein or nucleic acid is distinct from the way it exists in nature. Thus, for example, purified cDNA obtained by RT-PCR, or antibody captured polypeptides or purified polypeptides are contemplated herein. Such nucleic acids, polypeptide, antibodies etc. can be detectably labeled for optical measurements, radioisotope measurements etc. Such detectable labels do not “naturally occur” on such polypeptide, nucleic acid, antibodies and the like.

As used herein, the term “mass spectrometer” refers to a device able to volatilize/ionize analytes to form gas-phase ions and determine their absolute or relative molecular masses. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

The term “metabolite” refers to any substance produced by or transmutated in a metabolic reaction. A “metabolite” is considered to be in or belong to a particular metabolic pathway if it is a precursor, product, and/or intermediate of the pathway and/or if the pathway's precursor or product is readily traceable to the metabolite. Such a metabolite can be an organic compound that is a starting material, an intermediate in, or an end product of the metabolic pathway. Metabolites include molecules that during metabolism are used to construct more complex molecules and/or that are broken down into simpler ones. The term includes end products and intermediate metabolites.

In some embodiments, the presence and/or amount(s)/level(s) of specific metabolite(s) in a given metabolic pathway (e.g. products or intermediates of the pathway), and/or collections of such metabolites, are detected or measured, for example, by mass spectrometry and/or chromatography. In some embodiments, such detected amounts are compared to normal or control amounts. In some embodiments, the detected amounts are used to assess or detect alterations in the metabolic pathway, which in some aspects is informative for diagnosis and/or prediction of disease(s) or condition(s).

The terms “patient”, “subject” and “individual” are used interchangeably herein, and refer to an animal, particularly a human. This includes human and non-human animals. The term “non-human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. In a particular embodiment, the subject is a female subject. In a further embodiment, the subject is an infant. In yet a further embodiment, the subject is a human infant subject. In a particular embodiment, the subject is an infant of any from premature (i.e., less than 37 weeks gestation) to 1 year of age. In a further embodiment, the subject is a pregnant human female subject having a gestational period between 32 to 36 weeks.

Preterm birth refers to delivery or birth at a gestational age less than 37 completed weeks. Other commonly used subcategories of preterm birth have been established and delineate moderately preterm (birth at 32 to 36 weeks of gestation), very preterm (birth at <32 weeks of gestation), and extremely preterm (birth at ≤28 weeks of gestation). Gestational age is a proxy for the extent of fetal development and the fetus's readiness for birth. Gestational age has typically been defined as the length of time from the date of the last normal menses to the date of birth. However, obstetric measures and ultrasound estimates also can aid in determining gestational age. Preterm births have generally been classified into two separate subgroups. One, spontaneous preterm births are those occurring subsequent to spontaneous onset of preterm labor or preterm premature rupture of membranes regardless of subsequent labor augmentation or cesarean delivery. Two, indicated preterm births are those occurring following induction or cesarean section for one or more conditions that the woman's caregiver determines to threaten the health or life of the mother and/or fetus.

As used herein, a “risk indicator” refers to a factor that is predictive for infant mortality and/or morbidity in preterm and infant subjects. Risk indicators comprise various metabolites described herein, wherein the measured level of the metabolites is indicative of an infant's risk for mortality and morbidity. Risk indicators may also comprise gestational age, and birth weight. A more complete listing of risk indicators is further provided herein.

In the United States, approximately 1 out of every 10 live born infants is delivered preterm (before 37 weeks), and globally, the burden of preterm births stands at nearly 15 million per year. Preterm birth and related complications are the leading cause of death for children under 5 years of age, and neonatal deaths, specifically, account for 46% of mortality in this age group.

Beyond mortality, preterm infants are also at an increased risk for numerous complications including patent ductus arteriosus (PDA), respiratory distress syndrome (RDS), intraventricular hemorrhage (IVH), periventricular leukomalacia (PVL), bronchopulmonary dysplasia (BPD), retinopathy of prematurity (ROP), necrotizing enterocolitis (NEC), jaundice, infections, sepsis, and longer term, cerebral palsy and neurodevelopmental disability.

In the case of Sudden Infant Death Syndrome (SIDS) approximately 2000 infant deaths per year are attributed to SIDS. The majority of those deaths occur at age 1-4 months.

Cumulatively, these conditions result in increased health care resource utilization for infants costing at least 26.2 billion dollars each year in the United States. Additionally, infants continue to survive at increased frequency at lower gestational ages (GA) and birthweights (BW) due primarily to improvements in resuscitation and early neonatal care, which leads to reduced mortality but increased morbidity amongst extremely preterm infants. The convergence of human loss and economic cost have made the use of novel strategies—beyond insufficient models relying solely on gestational age, birthweight, and other common characteristics—to inform risk, minimize complications, and gain insight into underlying dysfunctional biological processes an increasingly critical priority.

Routine newborn screening (NBS) is typically utilized for identifying infants at risk for rare metabolic disorders, but has also begun to shed light on the contribution of metabolomics in evaluating the complications associated with prematurity. For example, researchers have demonstrated associations between amino acid concentrations and RDS, as well as PDA in preterm infants. Additional researchers have established associations between NBS metabolites and sepsis. Strong associations between measured NBS metabolites and persistent pulmonary hypertension, hyperbilirubinemia, NEC, and survival in extremely preterm (<26 GA weeks) infants have also been shown. Further understanding of how metabolic dysfunction in preterm infants contributes to the development of common complications could direct preventative treatments in the future.

It is known that preterm infants are at increased risk for mortality and major morbidity compared to their term counterparts. Even within preterm cohorts, there are infants who thrive and those who do not. These differences are often not explained by gestational age, birth weight, or other characteristics of the mother or infant alone. The studies presented herein established a relationship between initial metabolic profile and mortality and major morbidity in preterm infants. The newborn metabolic vulnerability profile disclosed herein was able to identify preterm infants that were more likely to experience at least one of these outcomes. The newborn metabolic vulnerability profile disclosed herein outperformed models based only on clinical characteristics like GA and BW. Furthermore, metabolic markers were identified that are especially useful for follow-up investigation into etiologic drivers of mortality and of specific complications. The newborn metabolic vulnerability profile disclosed herein can be used for targeted long-term clinical monitoring and interventions that are specific to a newborn's metabolic profile to improve both survival rates and long-term health outcomes for this highly vulnerable population. In addition, the profiles identified herein for risk of preterm infants was extended to analyze a post-term infants and their risk for SIDS.

The findings presented herein clearly establish that NBS metabolites provide additional utility beyond gestational age and birthweight. While older gestational age and increased birthweight were consistently associated with decreased risk for morbidity or mortality, the newborn metabolic vulnerability profile model disclosed herein outperformed the clinical characteristics model (GA and BW included) across groups. In addition to the identification of NBS metabolites for the newborn metabolic vulnerability profile model disclosed herein, the observed metabolite patterns appear to point to several potentially important etiologic pathways that could prove important for further clinical and investigative follow-up aimed at preventing mortality or major morbidity in infants born preterm. For example, in the studies presented herein elevated levels of TSH, a hormone indicative of thyroid function, were found to be associated with a lower risk of morbidity and mortality. The findings presented herein are supported by other studies examining TSH levels in preterm infants wherein it has been suggested that higher TSH levels may be related to a greater production of surfactant, which has been shown to be crucial in preventing or minimizing the severity of RDS. TSH and thyroid hormones are important in normal neonatal physiology, influencing everything from metabolic homeostasis to proper neurodevelopment. The importance of TSH in multiple physiologic pathways likely explains the significant role that TSH played in the newborn metabolic vulnerability model and in observed patterning across outcomes.

The confluence of raised concentrations of phenylalanine and lower levels of tyrosine being associated with increased risk of morbidity and mortality in the newborn metabolic vulnerability profile model disclosed herein may suggest a dysfunction in the biosynthesis of tyrosine from phenylalanine by phenylalanine hydroxylase (PAH) in these infants. In order to function properly, PAH relies on the cofactor tetrahydrobiopterin (BH₄), but BH₄ becomes depleted in situations of high oxidative stress due to inflammation and immune activation. Oxidative stress, in particular has been implicated in a number of neonatal outcomes including IVH, BPD, and RDS.

Lower levels of the amino acid ornithine were also associated with increased risk of complications. Ornithine, citrulline and arginine are all important intermediates within the urea cycle, which has been connected to neonatal outcomes including NEC and persistent pulmonary hypertension. It's hypothesized that reduced levels of urea cycle enzymatic activity, especially in carbamoyl-phosphate synthetase (responsible for catalyzing the rate dependent step) results in diminished concentrations of citrulline, arginine, and ornithine. Without these intermediates, ammonia can begin to accumulate and precipitate nervous and respiratory sequelae and potentially death.

Another significant metabolic pattern for the newborn metabolic vulnerability profile model disclosed herein was the relevance of short-chain acylcarnitines to the measured infant outcomes. Specifically, high levels of C-2 were generally associated with lower risk of morbidity and mortality while high levels of C-3, C-4, and C-5 were consistently associated with higher risk. Acylcarnitines are heavily involved in the process of shuttling fatty acids across the mitochondrial membrane in order to be utilized in β-oxidation. Therefore, abnormal levels of acylcarnitines may suggest systemic dysfunction of fatty acid oxidation.

The newborn metabolic vulnerability profile model disclosed herein was derived from an extensive and diverse population-based data set, minimizing the potential for significant selection bias. Furthermore, the data was split into training and validation sets to ensure the newborn metabolic vulnerability profile model was not over-fitted. Use of point of care technology for the newborn metabolic vulnerability profile model disclosed herein can be used to assess metabolic changes over time and deliver expedited results for potential real-time clinical decision-making. Further, the newborn metabolic vulnerability profile model disclosed herein can also be expanded to utilize untargeted metabolomics and machine learning with expanded sets of metabolites.

Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection.

Chromatography, such as gas chromatography (GC) and high-pressure liquid chromatography (HPLC), in some embodiments is used in the process of detecting and quantifying (e.g., detecting an amount of) one or more metabolites.

For example, in some embodiments, High Performance Liquid Chromatography (HPLC) is used in a method for identifying and/or separating a metabolite. HPLC columns equipped with coulometric array technology can be used to analyze the samples, separate the compounds, and/or create a metabolite profiles of the samples. HPLC columns are known and have been used in serum, urine and tissue analysis and are suitable for small molecule analysis (Beal et al., J Neurochem., 55:1327-1339, 1990; Matson et al., Life Sci., 41:905-908, 1987; Matson et al., Basic, Clinical and Therapeutic Aspects of Alzheimer's and Parkinson's Diseases, vol II, pp. 513-516, Plenum, N.Y. 1990; LeWitt et al., Neurology, 42:2111-2117, 1992; Ogawa et al., Neurology, 42:1702-1706, 1992; Beal et al., J. Neurol. Sci., 108:80-87, 1992; Matson et al., Clin. Chem., 30:1477-1488, 1984; Milbury et al., Coulometric Electrode Array Detectors for HPLC, pp. 125-141, VSP International Science Publication; Acworth et al., Am. Lab, 28:33-38, 1996).

In GC, the sample to be analyzed is introduced via a syringe into a narrow bore (capillary) column which sits in an oven. The column, which typically contains a liquid adsorbed onto an inert surface, is flushed with a carrier gas such as helium or nitrogen. In a properly set up GC system, a mixture of substances introduced into the carrier gas is volatilized, and the individual components of the mixture migrate through the column at different speeds. Detection takes place at the end of the heated column and is generally a destructive process. Very often the substance to be analyzed is “derivatized” to make it volatile or change its chromatographic characteristics. In contrast, for HPLC a liquid under high pressure is used to flush the column rather than a gas. Typically, the column operates at room or slightly above room temperature.

In some embodiments, Mass Spectroscopy (MS) Detectors are used in the identification and/or quantification of the metabolites. The sample, fraction thereof, compound, and/or molecule generally is ionized and passed through a mass analyzer where the ion current is detected. There are various methods for ionization. Examples of these methods of ionization include, but are not limited to, electron impact (EI) where an electric current or beam created under high electric potential is used to ionize the sample migrating off the column; chemical ionization utilizes ionized gas to remove electrons from the compounds eluting from the column; and fast atom bombardment where Xenon atoms are propelled at high speed in order to ionize the eluents from the column.

Gas chromatography/mass spectrometry (GC/MS) is a combination of two technologies. GC physically separates (chromatographs or purifies) the compound, and MS fragments provide a fingerprint of the chemical. Although sample preparation is extensive, using the methods together can improve accuracy, sensitivity, and/or specificity. The combination is sensitive (i.e., can detect low levels) and is specific. Furthermore, assay sensitivity can be enhanced by treating the test substance with reagents.

Liquid chromatography/mass spectrometry (LC/MS) is a combination of liquid chromatography methods and mass spectrometry methods. Liquid chromatography such as HPLC, when coupled with MS, provides improved accuracy, specificity, and/or sensitivity, for example, in detection of substances that are difficult to volatilize.

In some embodiments, Pyrolysis Mass Spectrometry can be used to identify and/or quantify metabolites. Pyrolysis is the thermal degradation of complex material in an inert atmosphere or vacuum. It causes molecules to cleave at their weakest points to produce smaller, volatile fragments called pyrolysate. Curie-point pyrolysis is a particularly reproducible and straightforward version of the technique, in which the sample, dried onto an appropriate metal is rapidly heated to the Curie-point of the metal. A mass spectrometer can then be used to separate the components of the pyrolysate on the basis of their mass-to-charge ratio to produce a pyrolysis mass spectrum (Meuzelaar et al. 1982) which can then be used as a “chemical profile” or fingerprint of the complex material analyzed. The combined technique is known as pyrolysis mass spectrometry (PyMS).

In another embodiment, Nuclear Magnetic Resonance (NMR) can be used to identify and/or quantify metabolites. Certain atoms with odd-numbered masses, including H and nC, spin about an axis in a random fashion. When they are placed between poles of a strong magnet, the spins are aligned either parallel or anti-parallel to the magnetic field, with parallel orientation favored since it is slightly lower energy. The nuclei are then irradiated with electromagnetic radiation which is absorbed and places the parallel nuclei into a higher energy state where they become in resonance with radiation.

In yet another embodiment, Refractive Index (RI) can be used to identify and/or quantify metabolites. In this method, detectors measure the ability of samples to bend or refract light. Each metabolite has its own refractive index. For most RI detectors, light proceeds through a bi-modular flow to a photodetector. One channel of the flow-cell directs the mobile phase passing through the column while the other directs only the other directs only the mobile phase. Detection occurs when the light is bent due to samples eluting from the column, and is read as a disparity between the two channels. Laser based RI detectors have also become available.

In another embodiment, Ultra-Violet (UV) Detectors can be used to identify and/or quantify metabolites. In this method, detectors measure the ability of a sample to absorb light. This could be accomplished at a fixed wavelength usually 254 nm, or at variable wavelengths where one wavelength is measured at a time and a wide range is covered, alternatively Diode Array are capable of measuring a spectrum of wavelengths simultaneously. Sensitivity is in the 10⁻⁸ to 10⁻⁹ gm/ml range. Laser based absorbance or Fourier Transform methods have also been developed.

In another embodiment, Fluorescent Detectors can be used to identify and/or quantify metabolites. This method measures the ability of a compound to absorb then re-emit light at given wavelengths. Each compound has a characteristic fluorescence. The excitation source passes through the flow-cell to a photodetector while a monochromator measures the emission wavelengths. Sensitivity is in the 10⁻⁹ to 10⁻¹¹ gm/ml. Laser based fluorescence detectors are also available.

In yet another embodiment, Radiochemical Detection methods can be used to identify and/or quantify metabolites. This method involves the use of radiolabeled material, for example, tritium or carbon 14. It operates by detection of fluorescence associated with beta-particle ionization, and it is most popular in metabolite research. The detector types include homogeneous detection where the addition of scintillation fluid to column effluent causes fluorescence, or heterogeneous detection where lithium silicate and fluorescence by caused by beta-particle emission interact with the detector cell. Sensitivity is 10⁻⁹ to 10⁻¹⁰ gm/ml.

Electrochemical Detection methods can be used to identify and/or quantify metabolites. Detectors measure compounds that undergo oxidation or reduction reactions. Usually accomplished by measuring gains or loss of electrons from migration samples as they pass between electrodes at a given difference in electrical potential. Sensitivity of 10⁻¹² to 10⁻¹³ gms/ml.

Light Scattering (LS) Detector methods can be used to identify and/or quantify metabolites. This method involves a source which emits a parallel beam of light. The beam of light strikes particles in solution, and some light is then reflected, absorbed, transmitted, or scattered. Two forms of LS detection may be used to measure transmission and scattering.

Nephelometry, defined as the measurement of light scattered by a particular solution. This method enables the detection of the portion of light scattered at a multitude of angles. The sensitivity depends on the absence of background light or scatter since the detection occurs at a black or null background. Turbidimetry, defined as the measure of the reduction of light transmitted due to particles in solution. It measures the light scatter as a decrease in the light that is transmitted through particulate solution. Therefore, it quantifies the residual light transmitted. Sensitivity of this method depends on the sensitivity of the machine employed, which can range from a simple spectrophotometer to a sophisticated discrete analyzer. Thus, the measurement of a decrease in transmitted light from a large signal of transmitted light is limited to the photometric accuracy and limitations of the instrument employed.

Near Infrared scattering detectors operate by scanning compounds in a spectrum from 700-1100 nm. Stretching and bending vibrations of particular chemical bonds in each molecule are detected at certain wavelengths. This method offers several advantages; speed, simplicity of preparation of sample, multiple analyses from single spectrum and nonconsumption of the sample.

Fourier Transform Infrared Spectroscopy (FT-IR) can be used to identify and/or quantify metabolites. This method measures dominantly vibrations of functional groups and highly polar bonds. The generated fingerprints are made up of the vibrational features of all the sample components (Griffiths 1986). FT-IR spectrometers record the interaction of IR radiation with experimental samples, measuring the frequencies at which the sample absorbs the radiation and the intensities of the absorptions. Determining these frequencies allows identification of the sample's chemical makeup, since chemical functional groups are known to absorb light at specific frequencies. Both quantitative and qualitative analysis are possible using the FT-IR detection method.

Dispersive Raman Spectroscopy is a vibrational signature of a molecule or complex system. The origin of dispersive raman spectroscopy lies in the inelastic collisions between the molecules composing say the liquid and photons, which are the particles of light composing a light beam. The collision between the molecules and the photons leads to an exchange of energy with consequent change in energy and hence wavelength of the photon.

Immunoassay methods are based on an antibody-antigen reaction, small amounts of the drug or metabolite(s) can be detected. Antibodies specific to a particular drug are produced by injecting laboratory animals with the drug or human metabolite. These antibodies are then tagged with markers such as an enzyme (enzyme immunoassay, EIA), a radio isotope (radioimmunoassay, RIA) or a fluorescence (fluorescence polarization immunoassay, FPIA) label. Reagents containing these labeled antibodies can then be introduced into urine samples, and if the specific drug or metabolite against which the antibody was made is present, a reaction will occur.

A biological sample obtained from a subject can be prepared for use in one or more of the foregoing identification/detection methods. The biological sample, can be divided for multiple parallel measurements and/or can be enriched for a particularly type of metabolite(s). For example, different fractionation procedures can be used to enrich the fractions for small molecules. For example, small molecules obtained can be passed over several fractionation columns. The fractionation columns will employ a variety of detectors used in tandem or parallel to generate the metabolite profile.

For metabolite assessment, the metabolites disclosed herein may be quantified in a suitable biological sample obtained from the infant (e.g., preterm infant or full term), such as a blood or a serum sample. Quantification of metabolites in the sample may be performed by any method including the methods exemplified herein, or other methods known in the art. In a particular embodiment, a multiplex immunoassay is utilized to measure the metabolites described herein. For example, a multiplex bead immunoassay may be utilized, wherein sets of uniquely labeled and identifiable beads, each uniquely labeled bead targeted to a single metabolite, are used to simultaneously assay a sample for a panel of metabolites. Exemplary multiplex assay platforms include those described in U.S. Pat. No. 8,075,854, entitled “Microfluidic chips for rapid multiplex ELISA,” by Yang; United States Patent Publication Number US20020127740, entitled “Quantitative microfluidic biochip and method of use,” by Ho, and United States Patent Publication Number 20040241776, entitled “Multiplex enzyme-linked immunosorbent assay for detecting multiple analytes,” by Giester. An exemplary multiplex immunoassay is the Luminex XMAP™ or like system. Mass spectrometry techniques may be utilized to analyze biomarker presence and/or concentration in the sample. For example, tandem mass spectroscopy techniques can be employed, as known in the art. Other analytical approaches as described herein, can be used as well, such as enzymatic assays.

The attained risk indicator values for each of the metabolites, and optionally risk indicator values for secondary risk indicators, are then inputted to the predictive model. The predictive model may comprise any model based on the selected risk indicators, for example, a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, or other predictive model known in the art.

The predictive calculations of the model (as well as model generation steps described herein) may be carried out by any suitable digital computer. Suitable digital computers may include portable devices, laptop and desktop computers, cloud computing systems, etc., using any standard or specialized operating system, such as a Unix, Windows™ or Linux™ based operating systems. The computer will comprise software instructions coded on a non-transitory tangible computer-readable medium such as a memory drive or disk, which such instructions direct the calculations of model generation or predictive scoring.

When the values have been inputted to the processor, the predictive model will then calculate a risk score indicative of the subject's risk of experiencing one or more SIDS, pre-term complications and the like. This risk score may be retrieved from, transmitted from, displayed by or otherwise outputted by the computer.

As described herein, the metabolites described herein, as well as the secondary risk indicators (e.g., sex of infant, cesarean delivery, maternal education, maternal race/ethnicity, gestational age, and birthweight), are highly predictive of a infant's risk for mortality or morbidity. Accordingly, the disclosure further provides for integrated assays to simultaneously measure multiple risk indicators in a single sample, such as an assay kit. The assay kits described herein can be used to assess the levels of the metabolites disclosed herein that have been shown to have a high correlation for morbidity or mortality in infants. Such assay kits provide a “one stop” kit to assess the relevant metabolites in a biological sample, so that a risk assessment of the subject for morbidity or mortality is convenient and easily to quantify/assess. In a particular embodiment, the kit comprises, consists essentially of, or consists of at least the 19 metabolites described herein. In another embodiment, the kit is directed to the quantification of a subset of the at least 19 metabolites described herein.

The assay kit will comprise a plurality of detection/quantification tools specific to each metabolite detected by the kit. Many of the metabolites disclosed herein comprise amino acids or acylcarnitines, which may be detected by immunoassays or like technologies. The detection/quantification tools may comprise capture ligands of multiple types, each directed to the selective capture of a specific biomarker in the sample. The detection/quantification tools may comprise labeling ligands of multiple types, each directed to the selective labeling of a specific biomarker in the sample, for example, comprising enzymatic, fluorescent, or chemiluminescent labels for the quantification of target species. For example, the capture and/or labeling ligands may comprise antibodies (or fragments thereof), affibodies, aptamers, or other moieties that specifically bind to a selected biomarker. The assay kit may further comprise labeled secondary antibodies, for example comprising enzymatic, fluorescent, or chemiluminescent labels and associated reagents.

In one embodiment, the assay kit comprises a solid support to which one or more individually addressable patches of capture ligands are present, wherein the capture ligands of each patch are directed to a specific metabolite described herein. In another embodiment, individually addressable patches of absorbent or adsorbing material are present, onto which individual aliquots of sample may be immobilized. Solid supports may include, for example, a chip, wells of a microtiter plate, a bead or resin. The chip or plate of the kit may comprise a chip configured for automated reading, as is known in the art.

In yet another embodiment, the assay kits of the disclosure comprise reagents or enzymes which create quantifiable signals based on concentration dependent reactions with biomarker species in the sample. Assay kits may further comprise elements such as reference standards of the biomarkers to be measured, washing solutions, buffering solutions, reagents, printed instructions for use, and containers.

Provided herein is a population-based retrospective cohort study that included all infants born in California between 2005 and 2011. Birth and outcomes data were obtained through the California Office of Statewide Health Planning and Development (OSHPD). This database maintains maternal admission and discharge records up to one year prior to birth and maternal and infant admission and discharge records up to one year after birth including birth certificate and death certificates. Metabolic data were collected as a part of routine newborn screening conducted by the California Department of Public Health. This program requires all newborns to have a heel-stick bloodspot taken between 12 hours and 7 days after delivery and has been described in extensive detail elsewhere (e.g., see Feuchtbaum et al., Genet Med 14:937-945; and Jelliffe-Pawlowski et al., Am J Obstet Gynecol 214:511 e511-513). The newborn screening data was linked to the OSHPD data using common variables.

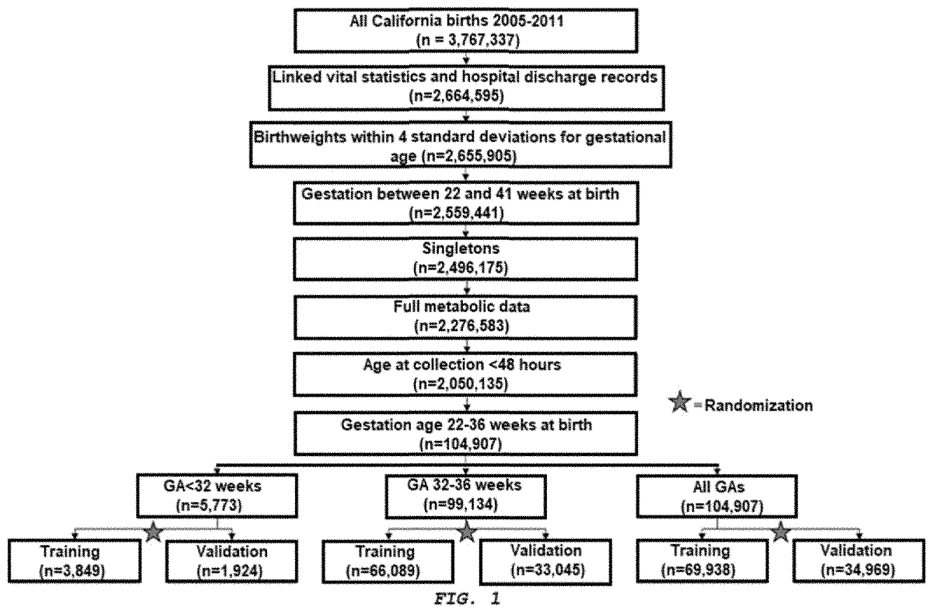

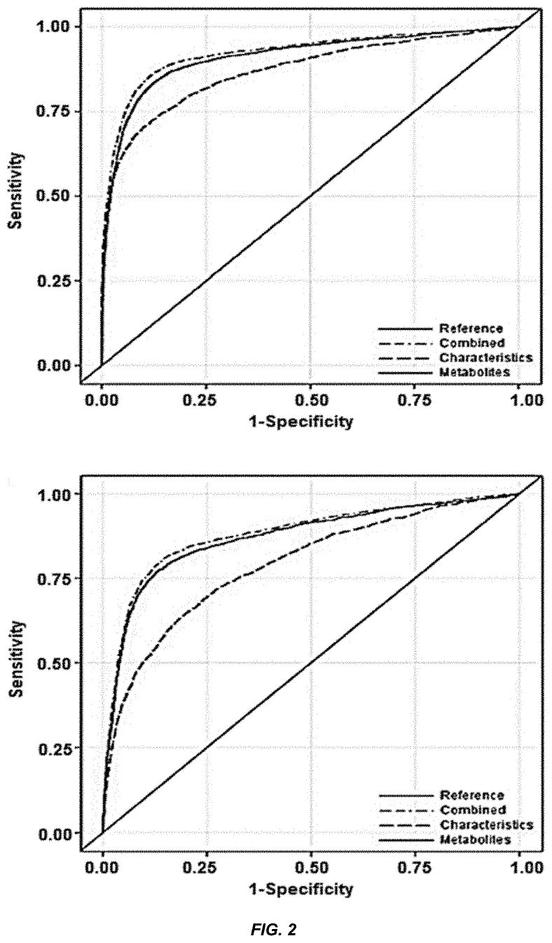

After linkage, 2,664,595 infants remained eligible for analysis. In the pre-term analysis, exclusion criteria included term birth (37 GA weeks), birthweight outside of four standard deviations from the mean for gestational age and sex, non-singleton birth, incomplete metabolic data measured by newborn screening, and blood spot collection after 48 hours. The final study sample consisted of 104,907 preterm infants. The cohort was split into gestational age groups (the full cohort, infants born at 32-36 weeks GA, and infants born at <32 weeks GA) and each group was randomly divided into a training set (⅔ of sample) and a validation set (⅓ of sample). Randomization was performed after stratification in order to ensure stratum likeness between training and validation groups (See FIG. 1).

Outcomes in the study were identified using the International Classification of Diseases 9th revision (ICD-9) diagnostic codes, linked infant death records, and hospital discharge status. The primary outcome was a composite measure that included infant death and major preterm morbidity. Infant death included neonatal death (within 28 days of birth) and one-year mortality. Preterm morbidities included: RDS (ICD-9 code 769.0), PDA (747.0), ROP (362.2), IVH (772.1), BPD (770.7), NEC (777.5), and PVL (779.7)]. It is believed that all deaths were captured given the linkage to death certificates and that ICD-9 codes captured ˜90% of all outcomes associated with preterm birth. Explanatory variables evaluated included infant and maternal characteristics from birth certificate and hospital discharge records as well as routine metabolites and clinical factors measured as part of NBS. Specific characteristics evaluated included infant gestational age at birth, birthweight, sex, small for gestational age (SGA), administration of total parenteral nutrition (TPN, defined as receiving TPN prior to blood spot collection), age at NBS collection, cesarean delivery, Medi-Cal status (California's Medicaid), adequacy of prenatal care, maternal race/ethnicity, maternal education, maternal age, gestational diabetes, and gestational hypertension. Variables were selected based on occurrence before NBS, availability, and previous or suspected relationship with the primary or secondary outcomes. Metabolites included from NBS consisted of 12 amino acids, 26 acylcarnitines, and free carnitine measured by standardized tandem mass spectrometry (MS/MS); two hormones measured by high-performance liquid chromatography; and one enzyme measured with a fluorometric enzyme assay (see Table 1).

TABLE 1
Crude analysis of maternal demographics, infant characteristics, and metabolites for
infants with a morbidity or mortality and those without in the training sample.

| | | | No | | | | |
|---|---|---|---|---|---|---|---|
| | Morbidity or | | Morbidity or | | | | |
| | Mortality | | Mortality | | OR | | |
| Variable | n = 6,404 | | n = 63,534 | | (95% CI) | | P-Value |
| Characteristics | | | | | | | |
| Gestational Age (weeks) | 31.3 | (3.7) | 35.3 | (1.2) | 0.48 | (0.48-0.49) | <0.0001 |
| Birthweight (per 100 g) | 17.9 | (8.1) | 28.3 | (5.2) | 0.77 | (0.76-0.77 | <0.0001 |
| Age at Collection (hours) | 31.0 | (9.5) | 29.0 | (8.6) | 1.03 | (1.02-1.03) | <0.0001 |
| Female | 2667 | (41.65) | 28951 | (45.57) | 0.85 | (0.81-0.90) | <0.0001 |
| TPN | 3513 | (54.86) | 2774 | (4.37) | 26.61 | (25.00-28.32) | <0.0001 |
| Small for GA | 802 | (12.52) | 3784 | (5.96) | 2.26 | (2.09-2.45) | <0.0001 |
| Cesarean delivery | 3823 | (59.7) | 19316 | (30.4) | 3.39 | (3.22-3.57) | <0.0001 |
| Medi-Cal | 3153 | (49.23) | 31638 | (49.8) | 0.98 | (0.93-1.03) | 0.3912 |
| Prenatal Care | | | | | | | <0.0001 |
| Adequate or adequate plus | 4932 | (77.01) | 49843 | (78.45) | | | |
| Intermediate | 555 | (8.67) | 4089 | (6.44) | 1.37 | (1.25-1.51) | |
| Inadequate | 676 | (10.56) | 7728 | (12.16) | 0.88 | (0.81-0.96) | |
| Unknown | 241 | (3.76) | 1874 | (2.95) | 1.30 | (1.13-1.49) | |
| Maternal Race | | | | | | | <0.0001 |
| White non-Hispanic | 1366 | (21.33) | 12932 | (20.35) | | | |
| Black | 389 | (6.07) | 2724 | (4.29) | 1.35 | (1.20-1.52) | |
| Hispanic | 3357 | (52.42) | 34429 | (54.19) | 0.92 | (0.86-0.99) | |
| Asian | 816 | (12.74) | 9425 | (14.83) | 0.82 | (0.75-0.90) | |
| Other | 476 | (7.43) | 4024 | (6.33) | 1.12 | (1.00-1.25) | |
| Maternal education | | | | | | | <0.0001 |
| <12 years | 1884 | (29.42) | 20605 | (32.43) | 0.88 | (0.83-0.94) | |
| 12 years | 1486 | (23.2) | 14427 | (22.71) | 1.00 | (0.93-1.06) | |
| >12 years | 2714 | (42.38) | 26224 | (41.28) | | | |
| unknown | 320 | (5) | 2278 | (3.59) | 1.36 | (1.20-1.54) | |
| Maternal age | | | | | | | <0.0001 |
| <18 years | 247 | (3.86) | 2497 | (3.93) | 1.02 | (0.90-1.17) | |
| 18-34 years | 4659 | (72.75) | 48229 | (75.91) | | | |
| >34 years | 1498 | (23.39) | 12808 | (20.16) | 1.21 | (1.14-1.29) | |
| Gestational diabetes | 741 | (11.57) | 6400 | (10.07) | 1.17 | (1.08-1.27) | 0.0002 |
| Gestational hypertension | 1088 | (16.99) | 6583 | (10.36) | 1.77 | (1.65-1.90) | <0.0001 |
| Enzymes & Hormones | | | | | | | |
| 17 Hydroxyprogesterone | 4.27 | (0.9) | 3.33 | (0.65) | 6.00 | (5.76-6.25) | <0.0001 |
| TSH | 0.74 | (0.96) | 1.47 | (0.61) | 0.24 | (0.23-0.25) | <0.0001 |
| GALT | 5.43 | (0.28) | 5.5 | (0.23) | 0.37 | (0.33-0.41) | <0.0001 |
| Amino Acids | | | | | | | |
| 5-Oxoproline | 3.37 | (0.61) | 3.54 | (0.55) | 0.65 | (0.62-0.68) | <0.0001 |
| Alanine | 5.5 | (0.42) | 5.48 | (0.33) | 1.19 | (1.10-1.28) | <0.0001 |
| Arginine | 2.66 | (0.92) | 2.15 | (0.68) | 2.62 | (2.53-2.72) | <0.0001 |
| Citrulline | 2.75 | (0.38) | 2.71 | (0.28) | 1.66 | (1.52-1.81) | <0.0001 |
| Glycine | 6.29 | (0.33) | 6.35 | (0.28) | 0.50 | (0.46-0.55) | <0.0001 |
| Leucine/Isoleucine | 4.94 | (0.49) | 4.65 | (0.28) | 12.13 | (11.24-13.09) | <0.0001 |
| Methionine | 3.55 | (0.63) | 3.34 | (0.33) | 3.92 | (3.68-4.17) | <0.0001 |
| Ornithine | 4.69 | (0.52) | 4.58 | (0.35) | 2.23 | (2.09-2.39) | <0.0001 |
| Phenylalanine | 4.37 | (0.32) | 4.15 | (0.23) | 25.55 | (23.05-28.31) | <0.0001 |
| Proline | 5.24 | (0.49) | 5.19 | (0.34) | 1.50 | (1.40-1.61) | <0.0001 |
| Tyrosine | 4.54 | (0.65) | 4.66 | (0.42) | 0.56 | (0.53-0.59) | <0.0001 |
| Valine | 4.87 | (0.46) | 4.67 | (0.31) | 6.42 | (5.94-6.94) | <0.0001 |
| Carnitines | | | | | | | |
| Free Carnitine | 3.77 | (0.46) | 3.57 | (0.41) | 3.01 | (2.83-3.21) | <0.0001 |
| C-2 | 3.17 | (0.37) | 3.2 | (0.31) | 0.76 | (0.70-0.83) | <0.0001 |
| C-3 | 0.89 | (0.43) | 0.69 | (0.4) | 3.29 | (3.08-3.50) | <0.0001 |
| C-3DC | −2.57 | (0.42) | −2.38 | (0.39) | 0.29 | (0.27-0.31) | <0.0001 |
| C-4 | −1.01 | (0.51) | −1.33 | (0.5) | 3.96 | (3.75-4.19) | <0.0001 |
| C-5 | −1.37 | (0.61) | −1.97 | (0.54) | 10.18 | (9.60-10.80) | <0.0001 |
| C-5:1 | −3.61 | (0.86) | −3.78 | (0.88) | 1.25 | (1.21-1.29) | <0.0001 |
| C-5DC | −2.17 | (0.48) | −2.06 | (0.45) | 0.59 | (0.56-0.63) | <0.0001 |
| C-5OH | −1.66 | (0.51) | −1.72 | (0.51) | 1.34 | (1.27-1.42) | <0.0001 |
| C-6 | −3 | (0.83) | −2.83 | (0.79) | 0.79 | (0.77-0.81) | <0.0001 |
| C-8 | −2.87 | (0.77) | −2.73 | (0.73) | 0.79 | (0.77-0.82) | <0.0001 |
| C-8:1 | −2.45 | (0.77) | −2.29 | (0.62) | 0.71 | (0.68-0.73) | <0.0001 |
| C-10 | −3.06 | (0.78) | −2.48 | (0.66) | 0.40 | (0.39-0.41) | <0.0001 |
| C-10:1 | −3.1 | (0.82) | −2.96 | (0.7) | 0.79 | (0.76-0.82) | <0.0001 |
| C-12 | −2.43 | (0.75) | −1.8 | (0.57) | 0.24 | (0.24-0.26) | <0.0001 |
| C-12:1 | −3.24 | (0.85) | −2.59 | (0.83) | 0.48 | (0.47-0.49) | <0.0001 |
| C-14 | −1.71 | (0.47) | −1.44 | (0.39) | 0.23 | (0.21-0.24) | <0.0001 |
| C-14:1 | −2.36 | (0.59) | −1.98 | (0.55) | 0.36 | (0.35-0.38) | <0.0001 |
| C-14OH | −4.05 | (0.83) | −3.74 | (0.84) | 0.67 | (0.65-0.69) | <0.0001 |
| C-16 | 0.68 | (0.47) | 0.98 | (0.34) | 0.12 | (0.11-0.13) | <0.0001 |
| C-16:1 | −1.77 | (0.52) | −1.53 | (0.46) | 0.39 | (0.37-0.41) | <0.0001 |
| C-16OH | −3.73 | (0.82) | −3.57 | (0.81) | 0.79 | (0.77-0.81) | <0.0001 |
| C-18 | −0.29 | (0.37) | −0.23 | (0.35) | 0.61 | (0.57-0.66) | <0.0001 |
| C-18:1 | −0.05 | (0.37) | 0.11 | (0.32) | 0.25 | (0.23-0.27) | <0.0001 |
| C-18:1OH | −4.02 | (0.81) | −3.89 | (0.83) | 0.83 | (0.81-0.86) | <0.0001 |
| C-18:2 | −1.34 | (0.63) | −1.6 | (0.51) | 2.74 | (2.60-2.89) | <0.0001 |
| C-18OH | −4.38 | (0.79) | −4.19 | (0.83) | 0.75 | (0.73-0.78) | <0.0001 |

TPN: total parenteral nutrition; NBS: newborn screening; GALT: galactose-1-phosphate uridyl transferase; TSH: thyroid stimulating hormone. Continuous variables described using mean and SD and ORs by per unit increase. Categorical variables described using frequencies and proportions. T-tests and χ² tests for continuous and categorical variables respectively

Statistical Analyses. In order to reduce skewness and minimize the influence of outliers, all metabolites were natural log transformed from their raw concentrations. Standardized growth curves by gestational age and sex were used to determine the 10th percentile cutoff to create SGA. Crude analyses to compare infants with any mortality or major morbidity to healthy infants relied on t-tests and χ² tests for continuous and categorical variables, respectively.

Multivariable logistic regression utilizing stepwise selection in the training dataset was used to create a metabolic vulnerability model for the composite outcome of mortality or any major morbidity. Any variable that met a Bonferonni corrected p-value threshold of <0.000781 was permitted to enter the model with entry order determined by greatest significance and a p-value of <0.05 required to remain in the model. Age at NBS collection and TPN were included into the model a-priori as they are known to affect the concentration of metabolites. The established model was then applied and tuned to mortality (neonatal and 1 year) and to each major morbidity (RDS, PDA, ROP, IVH, BPD, NEC, PVL) in the training and validation subsets with further evaluation of performance in preterm infants born at <32 and 32-36 weeks in the validation sample. Model performance was assessed using area under the curve (AUC) from a receiver operating characteristic (ROC) curve wherein variable importance was evaluated using odds ratios (ORs) with 95% confidence intervals (95% CI) and standardized beta coefficients. Multicollinearity between metabolites was examined by calculating Pearson correlation coefficients (0.8 considered strong collinearity) and by assessing the tolerance and variable inflation factors within the multivariable model (<0.1 and considered strong multicollinearity, respectfully). All analyses were performed using SAS 9.4 (SAS institute, Cary, NC).

Results. Within the population, 9,639 (9.2%) of infants with preterm birth experienced either mortality or at least one major complication. Training and validation samples had similar variable distributions. In univariable analysis, 13 of 14 infant and maternal characteristics as well as all 42 metabolites exhibited significant differences in preterm infants with mortality or major morbidity compared to those without (See Table 2; Table 1).

TABLE 2
Maternal demographics and infant characteristics in the training and validation sample.

| | Training | | | | | Validation | | | | |
|---|---|---|---|---|---|---|---|---|---|---|
| | Morbidity | | No Morbidity | | | Morbidity | | No Morbidity | | |
| | or Mortality | | or Mortality | | | or Mortality | | or Mortality | | |
| | n = 6,404 | | n = 63,534 | | P-val | n = 3,235 | | n = 31,734 | | P-val |
| Gestational Age (weeks) | 31.3 | (3.7) | 35.3 | (1.2) | <0.001 | 31.4 | (3.8) | 35.3 | (1.1) | <0.001 |
| Birthweight (per 100 grams) | 17.9 | (8.1) | 28.3 | (5.2) | <0.001 | 18.3 | (8.1) | 28.3 | (5.2) | <0.001 |
| Age at Collection (hours) | 31.0 | (9.5) | 29.0 | (8.6) | <0.001 | 30.7 | (9.6) | 29.0 | (8.6) | <0.001 |
| Female | 2667 | (41.65) | 28951 | (45.57) | <0.001 | 1337 | (41.33) | 14482 | (45.64) | <0.001 |
| TPN | 3513 | (54.86) | 2774 | (4.37) | <0.001 | 1715 | (53.01) | 1407 | (4.43) | <0.001 |
| Small for GA | 802 | (12.52) | 3784 | (5.96) | <0.001 | 399 | (12.33) | 1916 | (6.04) | <0.001 |
| Cesarean delivery | 3823 | (59.7) | 19316 | (30.4) | <0.001 | 1948 | (60.22) | 9794 | (30.86) | <0.001 |
| Medi-Cal | 3153 | (49.23) | 31638 | (49.8) | 0.391 | 1545 | (47.76) | 15892 | (50.08) | 0.012 |
| Prenatal Care | | | | | <0.001 | | | | | 0.068 |
| Adequate or adequate plus | 4932 | (77.01) | 49843 | (78.45) | | 2508 | (77.53) | 24912 | (78.5) | |
| Intermediate | 555 | (8.67) | 4089 | (6.44) | | 243 | (7.51) | 2078 | (6.55) | |
| Inadequate | 676 | (10.56) | 7728 | (12.16) | | 372 | (11.5) | 3795 | (11.96) | |
| Unknown | 241 | (3.76) | 1874 | (2.95) | | 112 | (3.46) | 949 | (2.99) | |
| Race | | | | | <0.001 | | | | | <0.001 |
| White | 1366 | (21.33) | 12932 | (20.35) | | 719 | (22.23) | 6298 | (19.85) | |
| Black | 389 | (6.07) | 2724 | (4.29) | | 187 | (5.78) | 1318 | (4.15) | |
| Hispanic | 3357 | (52.42) | 34429 | (54.19) | | 1661 | (51.34) | 17376 | (54.76) | |
| Asian | 816 | (12.74) | 9425 | (14.83) | | 432 | (13.35) | 4698 | (14.8) | |
| Other | 476 | (7.43) | 4024 | (6.33) | | 236 | (7.3) | 2044 | (6.44) | |
| Maternal education | | | | | <0.001 | | | | | <0.001 |
| <12 years | 1884 | (29.42) | 20605 | (32.43) | | 961 | (29.71) | 10422 | (32.84) | |
| 12 years | 1486 | (23.2) | 14427 | (22.71) | | 723 | (22.35) | 7118 | (22.43) | |
| >12 years | 2714 | (42.38) | 26224 | (41.28) | | 1407 | (43.49) | 13060 | (41.15) | |
| unknown | 320 | (5) | 2278 | (3.59) | | 144 | (4.45) | 1134 | (3.57) | |
| Maternal age | | | | | <0.001 | | | | | <0.001 |
| <18 years | 247 | (3.86) | 2497 | (3.93) | | 141 | (4.36) | 1261 | (3.97) | |
| 18-34 years | 4659 | (72.75) | 48229 | (75.91) | | 2323 | (71.81) | 23973 | (75.54) | |
| >34 years | 1498 | (23.39) | 12808 | (20.16) | | 771 | (23.83) | 6500 | (20.48) | |
| Gestational diabetes | 741 | (11.57) | 6400 | (10.07) | <0.001 | 363 | (11.22) | 3201 | (10.09) | 0.042 |
| Gestational hypertension | 1088 | (16.99) | 6583 | (10.36) | <0.001 | 535 | (16.54) | 3415 | (10.76) | <0.001 |

TPN: total parenteral nutrition;
GA: gestational age
Continuous variables described using mean and standard deviation and categorical variables using frequencies and proportions. T-tests and Chi-squared tests for continuous and categorical variables respectively were used to compare cases and controls

Among characteristics, gestational age, birthweight, SGA, cesarean delivery, and TPN were all highly associated with mortality or major morbidity. Metabolites strongly associated with mortality or major morbidity included high levels of 17-hydroxyprogesterone, leucine/isoleucine, phenylalanine, valine, and acylcarnitine C-5 and low levels of TSH and acylcarnitines C-12, C-14, C-16, and C-18:1 (see Table 1).

The multivariable metabolic vulnerability model for the composite outcome of any mortality or major morbidity included 6 characteristics (infant sex, cesarean delivery, maternal education, maternal race/ethnicity, gestational age, & birthweight) and 19 metabolites (three enzymes and hormones [17-hydroxyprogesterone, TSH, & GALT], seven amino acids [5-oxoproline, glycine, leucine/isoleucine, ornithine, phenylalanine, proline, & tyrosine], and nine acylcarnitines [C-2, C-3, C-4, C-5, C-10, C-12, C-12:1, C-16:1, & C-18:2]). The variables most strongly associated with any mortality or major morbidity included cesarean delivery (OR: 1.79; 95% CI: 1.66-1.93), gestational age (OR: 0.75; 95% CI: 0.73-0.77), birthweight (OR: 0.94; 95% CI: 0.93-0.95), 17-hydroxyprogesterone (OR: 1.61; 95% CI: 1.52-1.70), TSH (OR: 0.62; 95% CI: 0.59-0.65), ornithine (OR: 0.36; 95% CI: 0.32-0.42), phenylalanine (OR: 11.19; 95% CI: 9.03-13.87), tyrosine (OR: 0.53; 95% CI: 0.49-0.57), C-3 (OR: 1.42; 95% CI: 1.26-1.60), and C-12 (OR: 0.68; 95% CI: 0.64-0.73) (Table 3).

TABLE 3
Multivariable logistic regression model for any mortality or morbidity
adjusted for total parenteral nutrition and age at NBS collection.

| | Any | | No | | Parameter | | | |
|---|---|---|---|---|---|---|---|---|
| | Morbidity or | | Morbidity or | | Estimate | | OR | Standardized |
| Variable | Mortality | | Mortality | | (Std Error) | | (95% CI) | Estimate |
| Female | 2,667 | (41.65) | 28,951 | (45.57) | −0.31 | (0.04) | 0.74 (0.68-0.80) | −0.084 |
| C-Section | 3,823 | (59.70) | 19,316 | (30.40) | 0.58 | (0.04) | 1.79 (1.66-1.93) | 0.151 |
| Race/Ethnicity | | | | | | | | |
| White non-Hispanic | 1,366 | (21.33) | 12,932 | (20.35) | | | | |
| Asian | 816 | (12.74) | 9,425 | (14.83) | −0.35 | (0.06) | 0.71 (0.62-0.80) | −0.068 |
| Black | 389 | (6.07) | 2,724 | (4.29) | −0.13 | (0.09) | 0.87 (0.73-1.05) | −0.015 |
| Hispanic | 3,357 | (52.42) | 34,429 | (54.19) | −0.18 | (0.05) | 0.83 (0.75-0.92) | −0.051 |
| Other | 476 | (7.43) | 4,024 | (6.33) | −0.12 | (0.09) | 0.89 (0.75-1.05) | −0.017 |
| Education (years) | | | | | | | | |
| <12 | 1884 | (29.42) | 20,605 | (32.43) | −0.22 | (0.05) | 0.80 (0.73-0.89) | −0.057 |
| High School | 1,486 | (23.20) | 14,427 | (22.71) | −0.15 | (0.05) | 0.87 (0.78-0.96) | −0.034 |
| >12 | 2,714 | (42.38) | 26,224 | (41.28) | | | | |
| Unknown | 320 | (5.00) | 2,278 | (3.59) | −0.02 | (0.10) | 0.98 (0.81-1.19) | −0.002 |
| Birthweight (per 100 g) | 17.9 | (8.10) | 28.3 | (5.20) | −0.06 | (0.01) | 0.94 (0.93-0.95) | −0.216 |
| Gestational age (weeks) | 31.3 | (3.70) | 35.3 | (1.20) | −0.29 | (0.01) | 0.75 (0.73-0.77) | −0.311 |
| 17 ydroxyprogesterone | 4.27 | (0.90) | 3.33 | (0.65) | 0.47 | (0.03) | 1.61 (1.52-1.70) | 0.191 |
| TSH | 0.74 | (0.96) | 1.47 | (0.61) | −0.48 | (0.04) | 0.62 (0.59-0.65) | −0.179 |
| GALT | 5.43 | (0.28) | 5.50 | (0.23) | −0.47 | (0.08) | 0.63 (0.53-0.74) | −0.060 |
| 5-Oxoproline | 3.37 | (0.61) | 3.54 | (0.55) | −0.12 | (0.03) | 0.89 (0.83-0.95) | −0.037 |
| Glycine | 6.29 | (0.33) | 6.35 | (0.28) | 0.41 | (0.09) | 1.51 (1.27-1.78) | 0.064 |
| Leucine/Isoleucine | 4.94 | (0.49) | 4.65 | (0.28) | −0.54 | (0.11) | 0.59 (0.48-0.72) | −0.093 |
| Ornithine | 4.69 | (0.52) | 4.58 | (0.35) | −1.01 | (0.07) | 0.36 (0.32-0.42) | −0.205 |
| Phenylalanine | 4.37 | (0.32) | 4.15 | (0.23) | 2.42 | (0.11) | 11.19 (9.03-13.87) | 0.330 |
| Proline | 5.24 | (0.49) | 5.19 | (0.34) | 0.31 | (0.07) | 1.37 (1.19-1.57) | 0.062 |
| Tyrosine | 4.54 | (0.65) | 4.66 | (0.42) | −0.64 | (0.04) | 0.53 (0.49-0.57) | −0.159 |
| C-2 | 3.17 | (0.37) | 3.20 | (0.31) | −0.71 | (0.08) | 0.49 (0.42-0.58) | −0.121 |
| C-3 | 0.89 | (0.43) | 0.69 | (0.40) | 0.35 | (0.06) | 1.42 (1.26-1.60) | 0.078 |
| C-4 | −1.01 | (0.51) | −1.33 | (0.50) | 0.29 | (0.05) | 1.33 (1.22-1.45) | 0.081 |
| C-5 | −1.37 | (0.61) | −1.97 | (0.54) | 0.25 | (0.05) | 1.29 (1.17-1.41) | 0.079 |
| C-10 | −3.06 | (0.78) | −2.48 | (0.66) | −0.13 | (0.03) | 0.87 (0.83-0.92) | −0.051 |
| C-12 | −2.43 | (0.75) | −1.80 | (0.57) | −0.38 | (0.03) | 0.68 (0.64-0.73) | −0.130 |
| C-12:1 | −3.24 | (0.85) | −2.59 | (0.83) | −0.10 | (0.03) | 0.90 (0.86-0.95) | −0.048 |
| C-16:1 | −1.77 | (0.52) | −1.53 | (0.46) | 0.25 | (0.05) | 1.28 (1.17-1.42) | 0.064 |
| C-18:2 | −1.34 | (0.63) | −1.60 | (0.51) | −0.23 | (0.04) | 0.80 (0.74-0.86) | −0.066 |

All metabolites are natural log transformed. Original units - umol/L
GALT: galactose-1-phosphate uridyl transferase; TSH: thyroid stimulating hormone; OR: Odds Ratio; CI: confidence interval.
All variables have model p-value of <0.001
Continuous variables described using mean and SD and ORs by per unit increase. Categorical variables described using frequencies and proportions. T-tests and χ² tests for continuous and categorical variables respectively

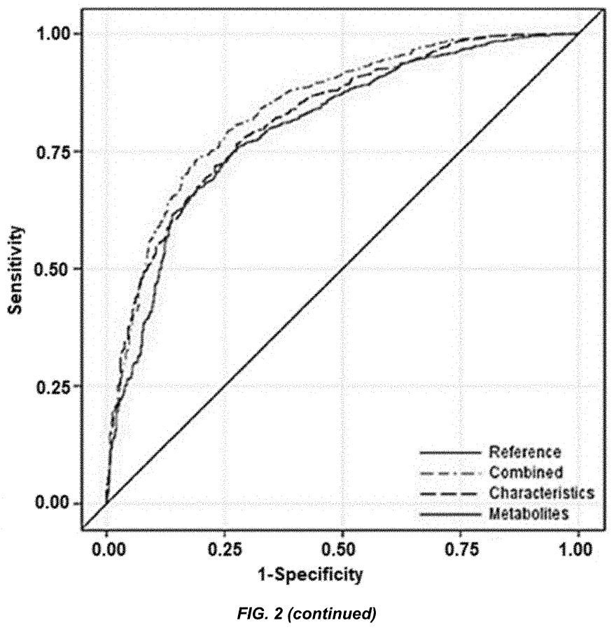

The metabolic vulnerability profile demonstrated exceptional performance in the validation sample (AUC 0.923 (0.917-0.929); Table 4), and at a probability cut point of 0.5, had 52.2% (95% CI: 50.5-53.9%) sensitivity, 98.4% (95% CI: 98.4-98.5%) specificity, 76.7% (95% CI: 74.9-78.5%) positive predictive value, and 95.3% (95.1-95.5%) negative predictive value (Table 5). The model also displayed robust performance when tuned to and evaluated by mortality and major complication subgroups (AUCs 0.893-0.977 across training and validation samples, Tables 6 and 7). Performance was maintained in groupings by gestational weeks at birth (<32 and 32-36 weeks, AUCs 0.682-0.929) with generally stronger performance observed in late preterm infants (Table 4 and FIG. 2). Models relying solely on the metabolic portion of the final model outperformed those relying only on characteristics for all outcomes except ROP and PVL (Table 8). The model remained robust when stratified by TPN (No TPN: AUC 0.880 (95% CI: 0.872-0.889); with TPN: AUC 0.821 (95% CI: 0.811-0.832)).

TABLE 4
Performance of the full model stratified by gestational age categories for individual outcomes within the validation population.

| | All Gestational Ages (n = 34,969) | | | Gestational ages <32 (n = 1,924) | | | Gestational ages 32-36 (n = 33,045) | | |
|---|---|---|---|---|---|---|---|---|---|
| Model | n (%) | | AUC (95% CI) | n = (%) | | AUC (95% CI) | n = (%) | | AUC (95% CI) |
| Morbidity or Mortality | 3,235 | (9.25) | 0.923 (0.917-0.929) | 1,485 | (77.18) | 0.841 (0.819-0.862) | 1,769 | (5.35) | 0.881 (0.871-0.891) |
| Neonatal Mortality | 157 | (0.45)* | 0.948 (0.928-0.967) | 104 | (5.56)* | 0.843 (0.803-0.883) | 67 | (0.20)* | 0.872 (0.819-0.924) |
| 1-Year Mortality | 266 | (0.76) | 0.913 (0.893-0.934) | 159 | (8.26) | 0.830 (0.797-0.864) | 120 | (0.36) | 0.815 (0.767-0.863) |
| RDS | 2,367 | (6.77) | 0.954 (0.950-0.958) | 1,243 | (64.60) | 0.787 (0.766-0.809) | 1,165 | (3.53) | 0.928 (0.919-0.936) |
| PDA | 1,367 | (3.91) | 0.893 (0.882-0.904) | 767 | (39.86) | 0.783 (0.762-0.803) | 578 | (1.75) | 0.814 (0.794-0.835) |
| ROP | 616 | (1.76) | 0.977 (0.971-0.984) | 592 | (30.77) | 0.756 (0.734-0.779) | 69 | (0.21) | 0.929 (0.894-0.964) |
| IVH | 554 | (1.58) | 0.953 (0.944-0.963) | 431 | (22.40) | 0.741 (0.715-0.767) | 131 | (0.40) | 0.878 (0.841-0.916) |
| BPD | 423 | (1.21) | 0.953 (0.941-0.966) | 366 | (19.02) | 0.806 (0.784-0.828) | 66 | (0.20) | 0.815 (0.756-0.874) |
| NEC | 182 | (0.52) | 0.952 (0.939-0.964) | 150 | (7.80) | 0.694 (0.655-0.734) | 62 | (0.19 | 0.908 (0.863-0.954) |
| PVL | 36 | (0.10) | 0.938 (0.887-0.989) | 32 | (1.66) | 0.682 (0.602-0.762) | 9 | (0.03) | 0.844 (0.691-0.997) |

*Full sample n = 34,860, n = 32,992, & n = 1,869 for gestational age categories respectively
AUC: area under the curve;
RDS: respiratory distress syndrome;
PDA: patent ductus arteriosus;
ROP: retinopathy of prematurity;
IVH: intraventricular hemorrhage;
BPD: bronchopulmonary dysplasia;
NEC: necrotizing enterocolitis;
PVL periventricular leukomalacia

TABLE 5
Performance of the metabolic vulnerability model at various probability cut points.

| | Any Morbidity | | | | | | |
|---|---|---|---|---|---|---|---|
| | or Mortality | No Morbidity or | Odds Ratio | Sensitivity | Specificity | PPV* | NPV* |
| | (n) | Mortality (n) | (95% CI) | (95% CI) | (95% CI) | (95% CI) | (95% CI) |
| Sample | 3,235 | 31,734 | | | | | |
| ≥0.9 | 816 | 38 | 1089.4 | 25.2 | 99.9 | 95.5 | 92.9 |
| | | | (778.8-1524.0) | (23.7-26.7) | (99.8-99.9) | (94.2-96.9) | (92.6-93.2) |
| ≥0.8 | 1103 | 107 | 522.9 | 34.1 | 99.7 | 91.2 | 93.7 |
| | | | (421.8-648.5) | (32.5-35.7) | (99.6-99.7) | (89.6-92.8) | (93.4-93.9) |
| ≥0.7 | 1313 | 206 | 323.4 | 40.6 | 99.4 | 86.4 | 94.3 |
| | | | (273.2-382.8) | (38.9-42.3) | (99.3-99.4) | (84.7-88.2) | (94.0-94.5) |
| ≥0.6 | 1509 | 336 | 227.8 | 46.6 | 98.9 | 81.8 | 94.8 |
| | | | (197.2-263.2) | (44.9-48.4) | (98.8-99.1) | (80.0-83.6) | (94.6-95.0) |
| ≥0.5 | 1688 | 513 | 166.9 | 52.2 | 98.4 | 76.7 | 95.3 |
| | | | (146.7-189.9) | (50.5-53.9) | (98.4-98.5) | (74.9-78.5) | (95.1-95.5) |
| ≥0.4 | 1885 | 744 | 128.5 | 58.3 | 97.7 | 71.7 | 95.8 |
| | | | (114.2-144.7) | (56.6-60.0) | (97.5-97.8) | (70.0-73.4) | (95.6-96.0) |
| ≥0.3 | 2104 | 1068 | 99.9 | 65.0 | 96.6 | 66.3 | 96.4 |
| | | | (89.5-111.7) | (63.4-66.7) | (96.4-96.8) | (64.7-68.0) | (96.2-96.7) |
| ≥0.2 | 2356 | 1628 | 73.4 | 72.8 | 94.9 | 59.1 | 97.2 |
| | | | (66.2-81.5) | (71.3-74.4) | (94.6-95.1) | (57.6-60.7) | (97.0-97.4) |
| <0.1 | 570 | 28918 | Reference | 82.4 | 91.1 | 48.6 | 98.1 |
| | | | | (81.1-83.7) | (90.8-91.4) | (47.3-50.0) | (97.9-98.2) |

**P-value < 0.001

TABLE 6
Multivariable logistic regression models with tuned parameters
for each individual outcome and gestational age stratification

| | MM | NM | 1yM | RDS | PDA | ROP | IVH | BPD | NEC | PVL |
|---|---|---|---|---|---|---|---|---|---|---|
| | Outcome All Gestational Ages | | | | | | | | | |
| Intercept | 7.281 | 0.428 | 0.842 | 4.104 | 5.153 | 5.568 | 5.849 | 8.141 | −2.914 | 0.671 |
| TPN* | 0.991 | 0.480 | 0.499 | 0.930 | 0.661 | 0.273 | 0.365 | 0.200 | 0.187 | 0.586 |
| Age at NBS Collection | 0.017 | −0.028 | −0.015 | 0.019 | 0.008 | 0.001 | 0.010 | −0.009 | 0.010 | 0.004 |
| Female | −0.306 | −0.266 | −0.349 | −0.360 | −0.060 | 0.046 | −0.364 | −0.350 | −0.261 | −0.030 |
| C-Section | 0.580 | 0.653 | 0.460 | 0.642 | 0.419 | 0.190 | −0.284 | 0.155 | 0.052 | 0.057 |
| Race/Ethnicity | | | | | | | | | | |
| Asian | −0.347 | 0.245 | 0.188 | −0.608 | −0.130 | −0.017 | 0.148 | −0.189 | 0.064 | −0.147 |
| White non-Hispanic | Ref | Ref | Ref | Ref | Ref | Ref | Ref | Ref | Ref | Ref |
| Black | −0.135 | −0.050 | 0.430 | −0.376 | −0.034 | −0.169 | −0.161 | −0.253 | 0.164 | 0.420 |
| Hispanic | −0.184 | 0.212 | 0.236 | −0.447 | 0.016 | 0.080 | −0.120 | −0.087 | −0.015 | 0.051 |
| Other | −0.122 | −0.201 | −0.119 | −0.344 | 0.053 | 0.358 | −0.013 | −0.092 | −0.830 | −0.143 |
| Education (years) | | | | | | | | | | |
| <12 | −0.219 | −0.182 | 0.027 | −0.359 | −0.107 | −0.021 | −0.065 | −0.211 | 0.237 | 0.258 |
| High School | −0.145 | 0.054 | 0.217 | −0.192 | −0.158 | 0.066 | −0.010 | −0.281 | −0.140 | 0.106 |
| >12 | Ref | Ref | Ref | Ref | Ref | Ref | Ref | Ref | Ref | Ref |
| Unknown | −0.021 | 0.002 | −0.076 | 0.074 | −0.463 | 0.222 | −0.385 | 0.131 | 0.074 | 0.525 |
| Birthweight (per 100 g) | −0.063 | −0.074 | −0.063 | −0.064 | −0.050 | −0.251 | −0.136 | −0.216 | −0.181 | −0.113 |
| Gestational age (weeks) | −0.288 | −0.070 | −0.084 | −0.218 | −0.206 | −0.155 | −0.168 | −0.152 | −0.032 | −0.166 |
| 17 Hydroxyprogesterone | 0.474 | 0.062 | 0.112 | 0.624 | 0.136 | 0.094 | 0.183 | 0.028 | 0.302 | 0.257 |
| TSH | −0.478 | −0.484 | −0.461 | −0.470 | −0.411 | −0.156 | −0.103 | −0.337 | −0.213 | −0.219 |
| GALT | −0.466 | −0.997 | −0.864 | −0.335 | −0.626 | 0.364 | −0.439 | −0.076 | 0.094 | 0.083 |
| 5-Oxoproline | −0.119 | −0.009 | −0.082 | −0.090 | −0.162 | 0.109 | −0.237 | −0.031 | −0.068 | 0.055 |
| Glycine | 0.410 | 0.995 | 0.905 | 0.290 | 0.547 | −0.028 | 0.012 | −0.281 | −0.164 | −0.675 |
| Leucine/Isoleucine | −0.537 | 0.579 | 0.087 | −0.526 | −0.296 | −0.734 | −0.473 | −0.403 | −0.542 | −1.206 |
| Ornithine | −1.012 | −1.819 | −1.503 | −0.943 | −1.011 | 0.133 | −0.526 | −0.142 | −0.241 | 0.266 |
| Phenylalanine | 2.415 | 0.938 | 1.215 | 2.414 | 1.634 | 0.887 | 0.877 | 0.769 | 1.250 | 0.591 |
| Proline | 0.313 | 0.449 | 0.375 | 0.191 | 0.375 | −0.215 | 0.809 | 0.132 | 0.285 | 1.180 |
| Tyrosine | −0.640 | −0.180 | −0.198 | −0.496 | −0.461 | −0.019 | −0.212 | −0.157 | −0.181 | −0.427 |
| C-2 | −0.705 | −0.302 | −0.276 | −1.025 | −0.376 | −0.714 | −0.569 | −0.463 | −0.477 | 0.361 |
| C-3 | 0.350 | −0.765 | −0.530 | 0.554 | 0.232 | 0.562 | 0.153 | 0.418 | 0.578 | −0.137 |
| C-4 | 0.285 | 0.607 | 0.313 | 0.265 | 0.316 | −0.103 | 0.164 | 0.304 | 0.022 | 0.769 |
| C-5 | 0.252 | 0.579 | 0.490 | 0.167 | 0.273 | 0.281 | 0.393 | −0.015 | −0.017 | −0.101 |
| C-10 | −0.134 | −0.277 | −0.164 | −0.167 | −0.020 | 0.074 | −0.094 | 0.032 | −0.120 | −0.041 |
| C-12 | −0.385 | −0.022 | −0.046 | −0.377 | −0.256 | −0.145 | −0.127 | −0.071 | −0.080 | −0.107 |
| C-12:1 | −0.102 | −0.117 | −0.080 | −0.096 | −0.031 | −0.092 | −0.191 | 0.037 | −0.093 | 0.187 |
| C-16:1 | 0.250 | 0.175 | 0.156 | 0.239 | 0.039 | 0.095 | 0.029 | −0.128 | −0.016 | 0.011 |
| C-18:2 | −0.227 | −0.092 | −0.029 | −0.206 | −0.104 | 0.141 | 0.019 | −0.043 | 0.054 | −0.242 |
| | Outcome <32 Weeks Gestation | | | | | | | | | |
| Intercept | 11.630 | 7.109 | 4.601 | 6.070 | 7.476 | 3.748 | 6.640 | 7.544 | −2.625 | −0.469 |
| TPN* | 0.276 | 0.232 | 0.099 | 0.251 | 0.209 | 0.092 | 0.002 | 0.080 | 0.064 | 0.488 |
| Age at NBS Collection | −0.001 | −0.015 | 0.001 | −0.006 | 0.003 | 0.003 | 0.004 | −0.012 | 0.013 | 0.012 |
| Female | −0.395 | 0.025 | −0.122 | −0.193 | −0.059 | −0.119 | −0.300 | −0.405 | −0.438 | 0.205 |
| C-Section | 0.169 | 0.134 | 0.144 | 0.115 | 0.178 | −0.028 | −0.405 | 0.099 | −0.078 | 0.174 |
| Race/Ethnicity | | | | | | | | | | |
| Asian | −0.351 | −0.197 | −0.032 | −0.592 | −0.034 | 0.106 | 0.162 | −0.099 | 0.217 | −0.108 |
| White non-Hispanic | Ref | Ref | Ref | Ref | Ref | Ref | Ref | Ref | Ref | Ref |
| Black | −0.229 | −0.499 | −0.096 | −0.310 | −0.396 | 0.045 | −0.236 | −0.489 | −0.126 | 0.174 |
| Hispanic | −0.399 | −0.087 | 0.006 | −0.557 | −0.189 | 0.029 | −0.105 | −0.148 | 0.165 | −0.346 |
| Other | −0.145 | −0.016 | 0.057 | −0.383 | −0.276 | 0.155 | 0.062 | −0.257 | −0.352 | −0.567 |
| Education (years) | | | | | | | | | | |
| <12 | −0.188 | −0.036 | −0.040 | −0.324 | −0.047 | 0.052 | −0.011 | −0.165 | 0.265 | 0.080 |
| High School | −0.286 | 0.442 | 0.370 | −0.188 | −0.253 | −0.118 | −0.055 | −0.281 | −0.016 | −0.302 |
| >12 | Ref | Ref | Ref | Ref | Ref | Ref | Ref | Ref | Ref | Ref |
| Unknown | −0.136 | 0.582 | 0.338 | 0.196 | −0.412 | −0.081 | −0.495 | −0.130 | −0.784 | 0.154 |
| Birthweight (per 100 g) | −0.198 | −0.089 | −0.113 | −0.141 | −0.126 | −0.192 | −0.055 | −0.221 | −0.137 | −0.017 |
| Gestational age (weeks) | −0.141 | −0.213 | −0.164 | −0.028 | −0.122 | −0.057 | −0.138 | −0.116 | −0.091 | −0.156 |
| 17 Hydroxyprogesterone | 0.367 | −0.006 | 0.005 | 0.310 | 0.149 | 0.065 | 0.140 | −0.046 | 0.168 | 0.078 |
| TSH | −0.501 | −0.138 | −0.121 | −0.345 | −0.364 | −0.165 | −0.129 | −0.296 | −0.176 | −0.250 |
| GALT | −1.125 | −0.858 | −0.618 | −0.728 | −0.606 | −0.017 | −0.633 | −0.084 | 0.219 | −0.264 |
| 5-Oxoproline | −0.332 | −0.046 | −0.052 | −0.159 | −0.062 | 0.040 | −0.246 | −0.042 | −0.147 | −0.058 |
| Glycine | 0.568 | 0.312 | 0.443 | 0.369 | 0.453 | 0.222 | 0.157 | −0.144 | 0.288 | −0.145 |
| Leucine/Isoleucine | 0.266 | 0.679 | 0.561 | 0.295 | −0.189 | −0.334 | −0.213 | −0.418 | 0.338 | −0.113 |
| Ornithine | −0.883 | −1.928 | −1.539 | −0.631 | −0.554 | 0.006 | −0.405 | 0.090 | −0.084 | −0.728 |
| Phenylalanine | 0.377 | 0.095 | 0.095 | 0.170 | 0.224 | 0.262 | 0.094 | 0.269 | −0.114 | 0.639 |
| Proline | 0.430 | 0.647 | 0.396 | 0.195 | 0.326 | −0.139 | 0.741 | 0.091 | 0.031 | 0.854 |
| Tyrosine | 0.007 | 0.173 | 0.123 | 0.006 | −0.005 | 0.089 | −0.029 | −0.054 | 0.039 | −0.163 |
| C-2 | −0.566 | 0.377 | 0.292 | −0.473 | −0.615 | −0.522 | −0.429 | −0.228 | −0.335 | 0.534 |
| C-3 | 0.449 | −0.619 | −0.492 | 0.318 | 0.286 | 0.353 | −0.104 | 0.321 | 0.132 | −0.350 |
| C-4 | 0.316 | 0.085 | 0.030 | 0.207 | 0.288 | −0.073 | 0.284 | 0.365 | −0.246 | 0.790 |
| C-5 | 0.164 | 0.154 | 0.230 | 0.062 | 0.226 | 0.175 | 0.244 | −0.030 | −0.159 | −0.189 |
| C-10 | −0.064 | −0.113 | −0.106 | −0.056 | 0.011 | 0.094 | 0.009 | 0.037 | −0.004 | 0.315 |
| C-12 | −0.026 | 0.051 | 0.051 | −0.055 | −0.058 | −0.075 | −0.006 | 0.012 | 0.168 | −0.235 |
| C-12:1 | −0.021 | 0.123 | 0.115 | 0.032 | −0.045 | −0.074 | −0.115 | −0.016 | −0.108 | 0.414 |
| C-16:1 | 0.041 | −0.096 | −0.101 | 0.057 | −0.074 | 0.140 | −0.041 | −0.171 | 0.064 | −0.049 |
| C-18:2 | −0.016 | −0.174 | −0.168 | 0.055 | 0.094 | 0.068 | −0.007 | 0.072 | −0.051 | −0.385 |
| | Outcome 32-36 Weeks Gestation | | | | | | | | | |
| Intercept | 1.524 | −5.740 | −3.131 | 2.998 | −5.310 | 7.555 | 9.972 | 15.604 | 4.350 | 20.735 |
| TPN* | 1.156 | 0.582 | 0.788 | 1.108 | 1.093 | 0.594 | 0.641 | 0.480 | 0.762 | 1.590 |
| Age at NBS Collection | 0.018 | −0.051 | −0.031 | 0.023 | 0.010 | −0.009 | −0.004 | −0.028 | 0.020 | −0.082 |
| Female | −0.297 | −0.257 | −0.253 | −0.425 | 0.027 | −0.423 | −0.065 | −0.673 | −0.056 | −0.040 |
| C-Section | 0.601 | 1.001 | 0.623 | 0.723 | 0.531 | 0.144 | 0.046 | 0.272 | −0.258 | −0.077 |
| Race/Ethnicity | | | | | | | | | | |
| Asian | −0.416 | 0.717 | 0.250 | −0.702 | −0.065 | −0.057 | 0.324 | −0.426 | 0.348 | 0.745 |
| White non-Hispanic | Ref | Ref | Ref | Ref | Ref | Ref | Ref | Ref | Ref | Ref |
| Black | −0.134 | 0.369 | 0.691 | −0.491 | 0.177 | 0.060 | −0.239 | −0.034 | −0.274 | −11.903 |
| Hispanic | −0.223 | 0.864 | 0.547 | −0.460 | 0.173 | 0.522 | −0.054 | 0.268 | 0.002 | 0.480 |
| Other | −0.142 | 0.028 | −0.137 | −0.368 | 0.335 | 0.829 | 0.274 | 0.049 | −0.920 | 0.409 |
| Education (years) | | | | | | | | | | |
| <12 | −0.249 | −0.106 | −0.125 | −0.404 | −0.075 | −0.066 | −0.057 | −0.097 | 0.338 | −0.788 |
| High School | −0.091 | −0.204 | 0.062 | −0.194 | −0.009 | −0.091 | 0.040 | 0.222 | 0.248 | −0.399 |
| >12 | Ref | Ref | Ref | Ref | Ref | Ref | Ref | Ref | Ref | Ref |
| Unknown | −0.101 | 0.030 | 0.079 | −0.029 | −0.541 | 0.189 | −0.221 | 0.749 | 0.712 | 0.529 |
| Birthweight (per 100 g) | −0.051 | −0.095 | −0.075 | −0.052 | −0.032 | −0.277 | −0.109 | −0.143 | −0.087 | −0.176 |
| Gestational age (weeks) | −0.185 | 0.102 | 0.061 | −0.241 | 0.000 | −0.077 | −0.376 | −0.094 | −0.273 | −0.269 |
| 17 Hydroxyprogesterone | 0.455 | −0.033 | 0.079 | 0.697 | −0.010 | 0.063 | 0.366 | −0.046 | 0.378 | −0.088 |
| TSH | −0.473 | −0.763 | −0.669 | −0.543 | −0.352 | −0.296 | −0.355 | −0.594 | −0.327 | 0.082 |
| GALT | −0.209 | −1.289 | −1.233 | −0.166 | −0.327 | 0.005 | −0.225 | −0.898 | −0.146 | −0.875 |
| 5-Oxoproline | −0.090 | −0.095 | −0.097 | −0.064 | −0.162 | 0.126 | −0.203 | −0.033 | −0.256 | 0.569 |
| Glycine | 0.500 | 0.857 | 0.907 | 0.389 | 0.594 | 0.319 | −0.270 | −0.379 | −1.014 | −1.795 |
| Leucine/Isoleucine | −0.554 | 1.601 | 0.579 | −0.616 | −0.563 | 0.522 | −0.522 | −0.289 | −0.774 | −1.977 |
| Ornithine | −1.117 | −2.361 | −1.827 | −1.079 | −1.240 | −0.941 | −0.545 | −0.130 | −1.135 | 0.853 |
| Phenylalanine | 2.557 | 1.643 | 1.606 | 2.678 | 2.428 | 0.477 | 1.169 | 1.128 | 1.931 | −0.459 |
| Proline | 0.240 | 0.315 | 0.406 | 0.131 | 0.557 | −0.797 | 0.841 | −0.520 | 1.181 | 1.792 |
| Tyrosine | −0.694 | −0.715 | −0.642 | −0.555 | −0.857 | −0.124 | −0.331 | −0.256 | −0.267 | −0.492 |
| C-2 | −0.731 | −0.170 | −0.129 | −1.165 | −0.310 | −0.481 | 0.186 | −1.311 | 0.469 | 0.083 |
| C-3 | 0.407 | −0.594 | −0.483 | 0.597 | 0.245 | −0.061 | −0.388 | 0.288 | 0.172 | −0.160 |
| C-4 | 0.301 | 1.394 | 0.778 | 0.270 | 0.316 | 0.247 | 0.287 | 0.493 | 0.253 | 0.567 |
| C-5 | 0.244 | 0.105 | 0.150 | 0.224 | 0.301 | 0.553 | 0.509 | 0.279 | 0.045 | −0.255 |
| C-10 | −0.165 | −0.327 | −0.096 | −0.189 | −0.080 | −0.027 | −0.090 | 0.078 | −0.012 | −0.415 |
| C-12 | −0.465 | −0.254 | −0.240 | −0.445 | −0.383 | −0.174 | −0.214 | 0.040 | −0.409 | −0.321 |
| C-12:1 | −0.117 | −0.036 | −0.060 | −0.134 | −0.033 | −0.124 | −0.034 | −0.215 | −0.125 | −0.168 |
| C-16:1 | 0.195 | −0.023 | −0.031 | 0.205 | 0.071 | 0.232 | −0.131 | 0.054 | −0.411 | 0.825 |
| C-18:2 | −0.254 | −0.381 | −0.060 | −0.313 | −0.169 | 0.046 | 0.017 | 0.154 | 0.344 | −0.025 |

*TPN given before NBS collection. All metabolites are log transformed. Original units—umol/L.
TPN: total parenteral nutrition;
NBS: newborn screening;
GALT: galactose-1-phosphate uridyl transferase;
TSH: thyroid stimulating hormone;
MM: mortality or morbidity;
NM: neonatal mortality;
1yM: 1-year mortality;
RDS: respiratory distress syndrome;
PDA: patent ductus arteriosus;
ROP: retinopathy of prematurity;
IVH: intraventricular hemorrhage;
BPD: bronchopulmonary dysplasia;
NEC: necrotizing enterocolitis;
PVL periventricular leukomalacia

TABLE 7
Performance of the model built for any morbidity or mortality applied
to individual outcomes in the training and testing datasets

| | Training (n = 69,938) | | | Validation (34,969) | | |
|---|---|---|---|---|---|---|
| Model | n = (%) | | AUC (95% CI) | n = (%) | | AUC (95% CI) |
| Morbidity or Mortality | 6,404 | (9.16) | 0.929 (0.925-0.933) | 3,235 | (9.25) | 0.923 (0.917-0.929) |
| Neonatal Mortality | 343 | (0.49)* | 0.948 (0.935-0.961) | 157 | (0.45)* | 0.948 (0.928-0.967) |
| 1-Year Mortality | 585 | (0.84) | 0.907 (0.891-0.922) | 266 | (0.76) | 0.913 (0.893-0.934) |
| RDS | 4,726 | (6.76) | 0.955 (0.952-0.958) | 2,367 | (6.77) | 0.954 (0.950-0.958) |
| PDA | 2,665 | (3.81) | 0.904 (0.896-0.911) | 1,367 | (3.91) | 0.893 (0.882-0.904) |
| ROP | 1,361 | (1.95) | 0.978 (0.974-0.982) | 616 | (1.76) | 0.977 (0.971-0.984) |
| IVH | 1,163 | (1.66) | 0.960 (0.954-0.966) | 554 | (1.58) | 0.953 (0.944-0.963) |
| BPD | 868 | (1.24) | 0.968 (0.961-0.975) | 423 | (1.21) | 0.953 (0.941-0.966) |
| NEC | 391 | (0.56) | 0.950 (0.939-0.960) | 182 | (0.52) | 0.952 (0.939-0.964) |
| PVL | 91 | (0.13) | 0.947 (0.920-0.975) | 36 | (0.10) | 0.938 (0.887-0.989) |

*Full Training n = 69,696 (other deaths not included) Testing n = 34,860
AUC: area under the curve;
RDS: respiratory distress syndrome;
PDA: patent ductus arteriosus;
ROP: retinopathy of prematurity;
IVH: intraventricular hemorrhage;
BPD: bronchopulmonary dysplasia;
NEC: necrotizing enterocolitis;
PVL periventricular leukomalacia

TABLE 8
Performance of the full model as compared to a model using only metabolites
and a model using only characteristics in the validation population

| | | | AUC (95% CI) | | |
|---|---|---|---|---|---|
| Model | n (%) | | Full | Metabolites | Characteristics |
| Morbidity or Mortality | 3,235 | (9.25) | 0.923 (0.917-0.929) | 0.911 (0.905-0.917) | 0.871 (0.863-0.879) |
| Neonatal Mortality | 157 | (0.45)* | 0.948 (0.928-0.967) | 0.938 (0.917-0.958) | 0.900 (0.871-0.930) |
| 1-Year Mortality | 266 | (0.76) | 0.913 (0.893-0.934) | 0.907 (0.887-0.928) | 0.873 (0.847-0.898) |
| RDS | 2,367 | (6.77) | 0.954 (0.950-0.958) | 0.943 (0.938-0.948) | 0.904 (0.897-0.911) |
| PDA | 1,367 | (3.91) | 0.893 (0.882-0.904) | 0.877 (0.865-0.889) | 0.853 (0.839-0.866) |
| ROP | 616 | (1.76) | 0.977 (0.971-0.984) | 0.964 (0.957-0.972) | 0.977 (0.971-0.984) |
| IVH | 554 | (1.58) | 0.953 (0.944-0.963) | 0.945 (0.935-0.954) | 0.943 (0.931-0.955) |
| BPD | 423 | (1.21) | 0.953 (0.941-0.966) | 0.949 (0.937-0.961) | 0.947 (0.933-0.961) |
| NEC | 182 | (0.52) | 0.952 (0.939-0.964) | 0.944 (0.929-0.959) | 0.931 (0.910-0.952) |
| PVL | 36 | (0.10) | 0.938 (0.887-0.989) | 0.934 (0.883-0.985) | 0.942 (0.903-0.982) |

*Full n = 34,860 (other deaths not included)
AUC: area under the curve;
RDS: respiratory distress syndrome;
PDA: patent ductus arteriosus;
ROP: retinopathy of prematurity;
IVH: intraventricular hemorrhage;
BPD: bronchopulmonary dysplasia;
NEC: necrotizing enterocolitis;
PVL periventricular leukomalacia

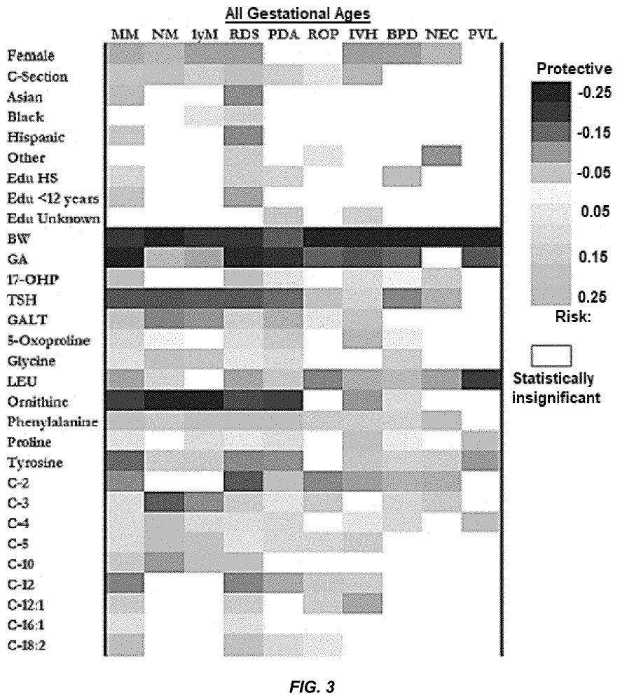

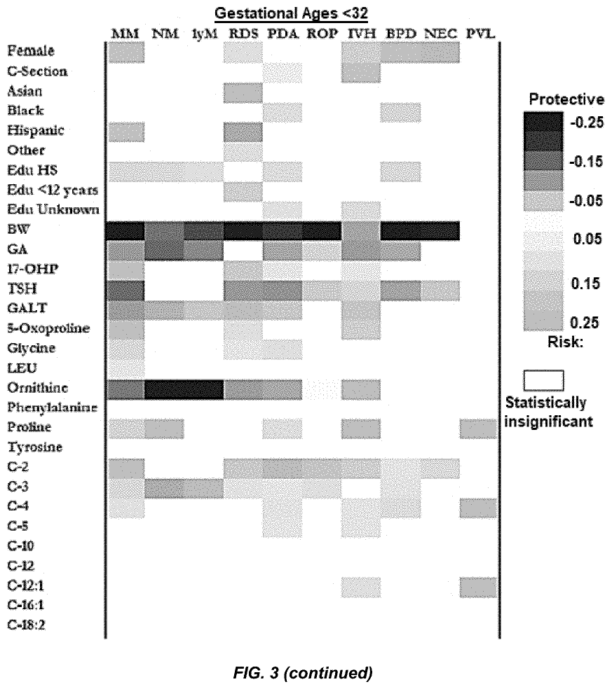

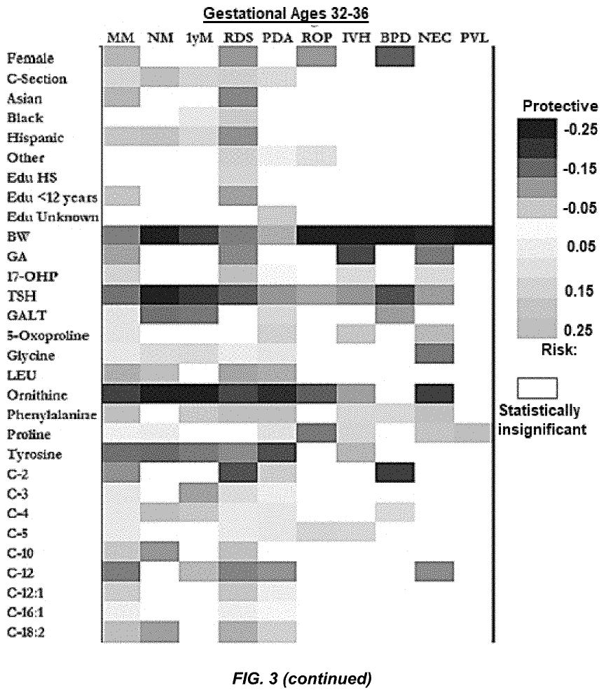

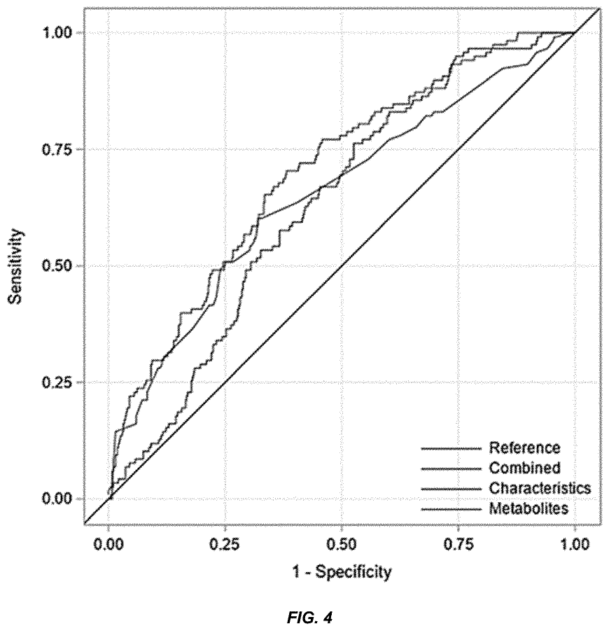

The importance of individual variables fluctuated within gestational age stratifications and by outcome (see FIG. 3). Birthweight was the only variable with ubiquitous importance across gestational age groups and outcomes as larger infants were less likely to experience morbidity or mortality. Older gestational age was also generally protective against morbidities and mortality. Female sex was associated with lower risk of mortality and most morbidities, and delivering via cesarean section was associated with increased risk of mortality, RDS, PDA, and ROP but decreased risk of IVH. Infants born between 32 and 36 weeks were at increased risk of mortality or morbidity if they had increased concentrations of phenylalanine, glycine, 17-OHP, proline, C-4, and C-5, and decreased concentrations of TSH, GALT, 5-oxoproline, ornithine, tyrosine, C-2, and C-12. For infants born before 32 weeks, increased risk for morbidity or mortality was associated with increased concentrations of 17-OHP, glycine, proline, and C-4 as well as decreased concentrations of TSH, GALT, 5-oxoproline, ornithine, and C-2 (see FIG. 3).

For SIDS analysis, the same data set as full metabolic vulnerability was used and included all gestational ages. Outcome was determined by ICD-9 code for SIDS cause of death (R95). Each SIDS outcome matched to 4 controls by birthweight z-score, which was broken down into 10 categories and gestational age. Matched data was divided into 2 groups: ⅔ of the set was applied to the training set and ⅓ was used for the testing set. Multivariable logistic regression using stepwise selection was used to build the model. All variables were allowed to enter and a p-value of less than 0.05 was necessary to remain in the model. Total parenteral (TPN) and age at collection were forced into the model as adjustment variables. Data was summarized using the same methods from the full metabolic vulnerability analyses.

TABLE 9
Multivariable logistic regression model for SIDS adjusted
for total parenteral nutrition and age at NBS collection

| | | | | Standardized | Odds Ratio |
|---|---|---|---|---|---|
| | Beta (std error) | | P-value | Beta | (95% CI) |
| Intercept | −6.0168 | (2.34) | 0.0100 | | |
| Total Parenteral Nutrition | −0.3601 | (0.49) | 0.4659 | 0.0372 | 0.70 (0.27-1.84) |
| Age at Collection (hours) | −0.0002 | (0.01) | 0.9717 | −0.0018 | 1.00 (0.99-1.01) |
| Prenatal Care | | | | | |
| Inadequate | 1.1395 | (0.22) | <0.0001 | 0.2001 | 3.13 (2.02-4.85) |
| Intermediate | 0.4023 | (0.24) | 0.0975 | 0.0692 | 1.50 (0.93-2.41) |
| Adequate & Adequate Plus | Reference | | Reference | Reference | Reference |
| Unknown | 0.348 | (0.50) | 0.4822 | 0.0307 | 1.42 (0.54-3.74) |
| Sex | | | | | |
| Male | Reference | | Reference | Reference | Reference |
| Female | −0.6638 | (0.17) | <0.0001 | −0.1825 | 0.52 (0.37-0.71) |
| Race/Ethnicity | | | | | |
| Asian | −0.6407 | (0.29) | 0.0263 | −0.1203 | 0.53 (0.30-0.93) |
| Black | 0.1909 | (0.34) | 0.5726 | 0.0235 | 1.21 (0.62-2.35) |
| Hispanic | −0.7469 | (0.20) | 0.0002 | −0.2059 | 0.47 (0.32-0.70) |
| White | Reference | | Reference | Reference | Reference |
| Other | −0.064 | (0.31) | 0.8384 | −0.0092 | 0.94 (0.51-1.74) |
| Maternal Age | | | | | |
| <18 | 0.7944 | (0.39) | 0.0430 | 0.0793 | 2.21 (1.03-4.78) |
| 18-34 | Reference | | Reference | Reference | Reference |
| >34 | −0.5909 | (0.25) | 0.0176 | −0.1244 | 0.55 (0.34-0.90) |
| Alanine | −1.4099 | (0.31) | <0.0001 | −0.2345 | 0.24 (0.13-0.45) |
| C-2 | 1.7956 | (0.38) | <0.0001 | 0.2964 | 6.02 (2.86-12.7) |
| C-3 | −0.5908 | (0.26) | 0.0211 | −0.1264 | 0.55 (0.34-0.92) |
| C-5DC | −0.6381 | (0.23) | 0.0061 | −0.1547 | 0.53 (0.34-0.83) |
| C-12:1 | −0.2933 | (0.10) | 0.0025 | −0.1416 | 0.75 (0.62-0.90) |
| C-14OH | 0.2319 | (0.10) | 0.0155 | 0.1093 | 1.26 (1.05-1.52) |
| C-18:1 | −0.6579 | (0.31) | 0.0366 | −0.1151 | 0.52 (0.28-0.96) |
| Glycine | 0.7998 | (0.34) | 0.0199 | 0.1192 | 2.23 (1.14-4.36) |
| 17 Hydroxyprogesterone | 0.3739 | (0.14) | 0.0074 | 0.135 | 1.45 (1.11-1.91) |

TABLE 10
Model AUCs including subset models using just metabolites
or just characteristics from the full model.

| Model | Training (2/3) | Testing (1/3) |
|---|---|---|
| Full | 0.759 (0.726-0.790) | 0.701 (0.650-0.750) |
| Metabolites | 0.701 (0.666-0.740) | 0.633 (0.582-0.680) |
| Characteristics | 0.668 (0.629-0.710) | 0.652 (0.594-0.710) |

TABLE 11
Performance of the final SIDS model at various probability cut points in training and testing sets.

| | | | | |
|---|---|---|---|---|
| SIDS | No SIDS | | | |
| n (95% CI) | n (95% CI) | Odds Ratio | Sensitivity | Specificity |
| % (95% CI) | % (95% CI) | (95% CI) | (95% CI) | (95% CI) |

Training

| Sample | 236 | (20.0) | 944 | (80.0) | | | | | |
|---|---|---|---|---|---|---|---|---|---|
| ≥0.5 | 40 | (32.2-47.8) | 26 | (18.2-33.8) | 26.3 | (13.5-51.3) | 66.7 | (54.7-78.6) | 92.9 (90.3-95.6) |
| | 60.6 | (48.8-72.4) | 39.4 | (27.6-51.2) | | | | | |
| ≥0.4 | 73 | (61.6-84.5) | 63 | (51.5-74.4) | 19.8 | (11.3-34.8) | 78.5 | (70.1-86.8) | 84.4 (80.9-88.0) |
| | 53.7 | (45.3-62.1) | 46.3 | (37.9-54.7) | | | | | |
| ≥0.3 | 106 | (90.6-121.3) | 141 | (125.7-156.4) | 12.9 | (7.7-21.5) | 84.1 | (77.8-90.5) | 70.8 (66.8-74.9) |
| | 42.9 | (36.7-49.1) | 57.1 | (50.9-63.3) | | | | | |
| ≥0.2 | 164 | (143.7-184.3) | 298 | (277.7-318.3) | 9.4 | (5.8-15.4) | 89.1 | (84.6-93.6) | 53.4 (49.6-57.3) |
| | 35.5 | (31.1-39.9) | 64.5 | (60.1-68.9) | | | | | |
| ≥0.1 | 216 | (191.4-240.5) | 602 | (577.5-626.6) | 6.1 | (3.8-9.9) | 91.5 | (88.0-95.1) | 36.2 (33.2-39.3) |
| | 26.4 | (23.4-29.4) | 73.6 | (70.6-76.6) | | | | | |
| <0.1 | 20 | (11.6-28.6) | 342 | (333.4-350.4) | Reference | | 91.5 | (88.0-95.1) | 36.2 (33.2-39.3) |
| | 5.5 | (3.2-7.9) | 94.5 | (92.1-96.8) | | | | | |

Testing

| Sample | 118 | (20.0) | 472 | (80.0) | | | | | |
|---|---|---|---|---|---|---|---|---|---|
| ≥0.5 | 17 | (11.5-22.5) | 15 | (9.5-20.5) | 12.0 | (5.0-28.8) | 14.4 | (8.1-20.7) | 96.8 (95.2-98.4) |
| | 53.1 | (35.8-70.4) | 46.9 | (29.6-64.2) | | | | | |
| ≥0.4 | 30 | (21.9-38.1) | 39 | (30.9-47.1) | 8.2 | (4.0-16.6) | 25.4 | (17.6-33.3) | 91.7 (89.3-94.2) |
| | 43.5 | (31.8-55.2) | 56.5 | (44.8-68.2) | | | | | |
| ≥0.3 | 48 | (36.9-59.1) | 94 | (82.9-105.1) | 5.4 | (2.9-10.2) | 40.7 | (31.8-49.5) | 80.1 (76.5-83.7) |
| | 33.8 | (26.0-41.6) | 66.2 | (58.4-74.0) | | | | | |
| ≥0.2 | 77 | (62.9-91.1) | 164 | (149.9-178.1) | 5.0 | (2.7-9.0) | 65.3 | (56.7-73.9) | 65.3 (61.0-69.6) |
| | 32.0 | (26.1-37.8) | 68.0 | (62.2-73.9) | | | | | |
| ≥0.1 | 103 | (85.7-120.2) | 313 | (295.8-330.3) | 3.5 | (2.0-6.2) | 87.3 | (81.3-93.3) | 33.7 (29.4-40.0) |
| | 24.8 | (20.6-28.9) | 75.2 | (71.1-79.4) | | | | | |
| <0.1 | 15 | (7.8-22.3) | 159 | (151.7-166.2) | Reference | | 87.3 | (81.3-93.3) | 33.7 (29.4-40.0) |
| | 8.6 | (4.5-12.8) | 91.4 | (87.2-95.5) | | | | | |

TABLE 12
Performance of the final SIDS model at various probability
cut point intervals in both training and testing sets

| | | |
|---|---|---|
| SIDS | No SIDS | |
| n (95% CI) | n (95% CI) | Odds Ratio |
| % (95% CI) | % (95% CI) | (95% CI) |

Training

| Sam- | 236 | (20.0) | 944 | (80.0) | | |
|---|---|---|---|---|---|---|
| ple | | | | | | |
| ≥0.5 | 40 | (32.2-47.8) | 26 | (18.2-33.8) | 26.3 | (13.5-51.3) |
| | 60.6 | (48.8-72.4) | 39.4 | (27.6-51.2) | | |
| 0.4- | 33 | (24.9-41.2) | 37 | (28.8-45.2) | 15.3 | (8.0-29.2) |
| 0.5 | 47.1 | (35.5-58.8) | 52.9 | (41.2-64.5 | | |
| 0.3- | 33 | (23.5-42.4) | 78 | (68.6-87.5) | 7.2 | (3.9-13.3) |
| 0.4 | 29.7 | (21.2-38.2) | 70.3 | (61.8-78.8) | | |
| 0.2- | 58 | (45.2-70.7) | 157 | (144.3-169.9) | 6.3 | (3.7-10.9) |
| 0.3 | 27.0 | (21.0-32.9) | 73.0 | (67.1-79.0) | | |
| 0.1- | 52 | (38.8-65.1) | 304 | (290.9-317.2) | 2.9 | (1.7-5.0) |
| 0.2 | 14.6 | (10.9-18.3) | 85.4 | (81.7-89.1) | | |
| <0.1 | 20 | (11.4-28.1) | 342 | (327.9-344.6) | Reference | |
| | 5.5 | (3.2-7.9) | 94.5 | (92.1-96.8) | | |

Testing

| Sam- | 118 | (20.0) | 472 | (80.0) | | |
|---|---|---|---|---|---|---|
| ple | | | | | | |
| ≥0.5 | 17 | (11.5-22.5) | 15 | (9.5-20.5) | 12.0 | (5.0-28.8) |
| | 53.1 | (35.8-70.4) | 46.9 | (29.6-64.2) | | |
| 0.4- | 13 | (7.3-18.7) | 24 | (18.3-29.7) | 5.7 | (2.4-13.5) |
| 0.5 | 35.1 | (19.8-50.5) | 64.9 | (49.5-80.2) | | |
| 0.3- | 18 | (10.8-25.2) | 55 | (47.8-62.2) | 3.5 | (1.6-7.3) |
| 0.4 | 24.7 | (14.8-34.5) | 75.3 | (65.5-85.2) | | |
| 0.2- | 29 | (20.1-37.9) | 70 | (61.1-78.9) | 4.4 | (2.2-8.7) |
| 0.3 | 29.3 | (20.3-38.3) | 70.7 | (61.7-79.7) | | |
| 0.1- | 26 | (16.8-35.2) | 149 | (139.8-158.2) | 1.8 | (0.9-3.6) |
| 0.2 | 14.9 | (9.6-20.1) | 85.1 | (79.9-90.4) | | |
| <0.1 | 15 | (7.8-22.3) | 159 | (151.7-166.2) | Reference | |
| | 8.6 | (4.5-12.8) | 91.4 | (87.2-95.5) | | |

It will be understood that various modifications may be made without departing from the spirit and scope of this disclosure. Accordingly, other embodiments are within the scope of the following claims.

What is claimed is:

1. A method of generating a risk assessment score for a biological sample obtained from a newborn infant, comprising:
measuring the level of a panel of metabolites in the sample, wherein the panel of metabolites comprises two or more the group consisting of thyroid stimulating hormone (TSH), galactose 1-phosphate uridylyltransferase (GALT), 17-hydroxyprogesterone (17-OHP), 5-oxoproline, glycine, leucine/isoleucine, ornithine, phenylalanine, proline, tyrosine, C-2 acylcarnitine, C-3 acylcarnitine, C-4 acylcarnitine, C-5 acylcarnitine, C-10 acylcarnitine, C-12 acylcarnitine, C-12:1 acylcarnitine, C-16:1 acylcarnitine, and C-18:2 acylcarnitine;
assigning a risk indicator value or predictor for each of the measured metabolites;
inputting the obtained risk indicator value into a computer-implemented predicative multivariate logistic model that is built using a training set and a testing set from a population of infants with any mortality or major morbidity to healthy infants; and
calculating a risk assessment score for the biological sample obtained from the newborn infant using the predicative multivariate logistic model.

2. The method of claim 1, wherein the newborn infant is a preterm infant.

3. The method of claim 2, wherein the sample is obtained from a preterm infant that is born at a gestation age of 32-36 weeks.

4. The method of claim 2, wherein the sample is obtained from a preterm infant that is born at a gestation age of under 32 weeks.

5. The method of claim 1, wherein the newborn infant is a full-term infant.

6. The method of claim 1, wherein the sample is a serum or a blood sample.

7. The method of claim 1, wherein the one or more metabolites are measured using tandem mass spectrometry (MS/MS), high-performance liquid chromatography, and/or a fluorometric enzyme assay.

8. The method of claim 1, wherein the predicative multivariate logistic model further includes risk indicator values or predictor values for one or more characteristics selected from sex of preterm infant, cesarean delivery, maternal education, maternal race/ethnicity, gestational age, and birthweight.

9. The method of claim 2, wherein the preterm infant is at higher risk for a morbidity selected from patent ductus arteriosus (PDA), respiratory distress syndrome (RDS), intraventricular hemorrhage (IVH), periventricular leukomalacia (PVL), bronchopulmonary dysplasia (BPD), retinopathy of prematurity (ROP), necrotizing enterocolitis (NEC), jaundice, infections, sepsis, longer term, cerebral palsy, and/or neurodevelopmental disability.

10. The method of claim 5, wherein the infant is at higher risk for Sudden Infant Death Syndrome (SIDS).

11. The method of claim 3, wherein the preterm infant is at a higher risk for morbidity or mortality if there is an increased measured concentration for phenylalanine, glycine, 17-OHP, proline, C-4 acylcarnitine, and C-5 acylcarnitine, and a decreased measured concentration for TSH, GALT, 5-oxoproline, ornithine, tyrosine, C-2 acylcarnitine, and C-12 acylcarnitine.

12. The method of claim 4, wherein the preterm infant is at a higher risk for morbidity or mortality if there is an increased measured concentration for 17-OHP, glycine, proline, and C-4 acylcarnitine and a decreased measured concentration for TSH, GALT, 5-oxoproline, ornithine, and C-2 acylcarnitine.

13. The method of claim 1, wherein the panel of metabolites are measured using a quantitative multiplex assay.

14. The method of claim 13, wherein the quantitative multiplex assay is a quantitative bead-based multiplex immunoassay.

15. The method of claim 1, wherein the predicative multivariate logistic model is a linear discriminant analysis model.

16. The method of claim 15, wherein the linear discriminant analysis model uses the coefficients for the biomarkers presented in Table 2 or 9.

17. The method of claim 1, wherein the predictive multivariate logistic model uses the coefficients for the biomarkers presented in Table 2 or 9.

18. The method of claim 1, further comprising:
clinical monitoring and investigating etiologic metabolic pathways of the preterm infant that are related or give rise to the morbidity/mortality predictive value generated from the metabolic vulnerability regression model.

19. A kit for assessing preterm birth and preeclampsia risk biomarkers in a sample, wherein the kit comprises a detecting agent(s) for each metabolite in a panel of metabolites consisting essentially of thyroid stimulating hormone (TSH), galactose 1-phosphate uridylyltransferase (GALT), 17-hydroxyprogesterone (17-OHP), 5-oxoproline, glycine, leucine/isoleucine, ornithine, phenylalanine, proline, tyrosine, C-2 acylcarnitine, C-3 acylcarnitine, C-4 acylcarnitine, C-5 acylcarnitine, C-10 acylcarnitine, C-12 acylcarnitine, C-12:1 acylcarnitine, C-16:1 acylcarnitine, and C-18:2 acylcarnitine.

20. The kit of claim 19, wherein the detecting agents are antibodies.

21. The kit of claim 20, wherein the kit is an ELISA or antibody microarray.
